(19) United States
(12) Patent Application Publication
(10) Pub. No.: US 2026/0028505 A1
(43) Pub. Date: Jan. 29, 2026

(54) 1,3-PROPANEDIOL ACRYLATES, DIACRYLATES, MONOACRYLATES, METHACRYLATES, AND DIGLYCIDYL ETHER AND USES THEREOF

(71) Applicant: Primient Covation LLC, Loudon, TN (US)

(72) Inventor: David Lof, Vellinge (SE)

(73) Assignee: Primient Covation LLC, Loudon, TN (US)

(21) Appl. No.: 19/281,117

(22) Filed: Jul. 25, 2025

Related U.S. Application Data
(60) Provisional application No. 63/675,360, filed on Jul. 25, 2024.

(51) Int. Cl. C09D 175/04; C07C 67/08; C07C 67/14; C07C 67/54; C07C 69/54; C07C 69/602; C07D 301/28; C07D 301/32; C07D 303/27

(52) U.S. Cl. CPC C09D 175/04; C07C 67/08; C07C 67/14; C07C 67/54; C07C 69/54; C07C 69/602; C07D 301/28; C07D 301/32; C07D 303/27

(57) ABSTRACT

Disclosed herein are processes for producing 1,3-propanediol acrylate, 1,3-propanediol diacrylate, 1,3-propanediol monoacrylate component, 1,3-propanediol dimethylacrylate, and 1,3-propanediol diglycidyl ether. Also disclosed herein are compositions comprising 1,3-propanediol acrylate, 1,3-propanediol diacrylate, 1,3-propanediol monoacrylate component, 1,3-propanediol dimethylacrylate, and/or 1,3-propanediol diglycidyl ether.

### CROSS-REFERENCE TO RELATED APPLICATIONS

This application claims the benefit of U.S. Provisional Application No. 63/675,360, filed Jul. 25, 2024, which is incorporated herein, in its entirety, by reference.

### FIELD

The present disclosure is in the field of polymer chemistry.

### BACKGROUND

Glycol acrylates, diacrylates, monoacrylates, methacrylates, and diglycidyl ethers are widely known and used monomers which are employed, in particular, for crosslinking. The glycol di(meth)acrylates, acrylates, and diglycidyl ethers have polymerizable vinyl group(s) in each molecule, so they are used in quite broad applications as starting materials or intermediates for thermosetting paints, adhesives, nonwoven fabric binders, photosensitive agents, paper finishing agents, copolymer modifiers and cross-linking agents, but the type of glycol must be selected carefully for obtaining the most suitable glycol acrylates, di(meth)acrylates, and/or diglycidyl ethers for each application.

There is a need, however, for improved glycol acrylates, di(meth)acrylates, and/or diglycidyl ethers for such applications.

### SUMMARY

In certain aspects, the disclosure relates to an adhesive or sealant comprising a 1,3-propanediol diacrylate component, 1,3-propanediol monoacrylate component, 1,3-propanediol dimethacrylate component, and/or a 1,3-propanediol di-glycidylether component.

In certain aspects, the disclosure relates to an ink comprising a 1,3-propanediol diacrylate component, and/or 1,3-propanediol monoacrylate component. In some embodiments, the ink is a flexographic ink, an inkjet ink, or a screen ink.

In certain aspects, the disclosure relates to a coating comprising a 1,3-propanediol diacrylate component, and/or 1,3-propanediol monoacrylate component. In some embodiments, the coating is a metal coating or a wood coating.

In certain aspects, the disclosure relates to an artificial marble comprising a 1,3-propanediol dimethacrylate component.

In certain aspects, the disclosure relates to a composite comprising a 1,3-propanediol dimethacrylate component.

In certain aspects, the disclosure relates to a coating comprising a 1,3-propanediol dimethacrylate component and/or a 1,3-propanediol di-glycidylether component. In some embodiments, the coating is a conformal coating or a wood coating. In some embodiments, the coating comprises a 1,3-propanediol di-glycidylether component and is a protective coating, a marine coating, or an electronics coating.

In certain aspects, the disclosure relates to an electrical potting composition comprising a 1,3-propanediol dimethacrylate component.

In certain aspects, the disclosure relates to an encapsulation composition comprising a 1,3-propanediol dimethacrylate component. In some embodiments, the encapsulation composition is a flooring and/or waterproofing encapsulation composition.

In certain aspects, the disclosure relates to a composite comprising a 1,3-propanediol di-glycidylether component.

In certain aspects, the disclosure relates to a reactive diluent comprising a 1,3-propanediol diacrylate component, 1,3-propanediol monoacrylate component, and/or a 1,3-propanediol di-glycidylether component.

In certain aspects, the disclosure relates to a coating composition comprising the reactive diluent of claim 1, a resin, and a reaction initiator. In some embodiments, the resin is an acrylic resin; and in some embodiments, the acrylic resin is a urethane acrylate. In some embodiments, the reaction initiator is a photoinitiator. In some embodiments, the coating composition has a chemical resistance of at least about 20 s. In some embodiments, the coating composition has a Persoz hardness of at least about 40%. In some embodiments, the coating composition has an impact resistance of at least about 950 mm on a substrate side an object coated with the coating composition.

In certain aspects, the disclosure relates to a coated article comprising the aforementioned coating composition on at least of portion of one surface of the article. In some embodiments, the coated article is a metal coated article or a wood coated article.

In certain aspects, the disclosure relates to a process for preparing a coating composition comprising mixing a resin with a reactive diluent comprising a 1,3-propanediol diacrylate component, 1,3-propanediol monoacrylate component, and/or a 1,3-propanediol di-glycidylether component; and reacting the resin with the reactive diluent in the presence of a reaction initiator. In some embodiments, the resin is an acrylic resin; and in some embodiments, the acrylic resin is a urethane acrylate. In some embodiments, the reaction initiator is a photoinitiator.

In certain aspects, the disclosure relates to a process for coating an article comprising mixing a resin, a reaction initiator, and a reactive diluent comprising a 1,3-propanediol diacrylate component, 1,3-propanediol monoacrylate component, and/or a 1,3-propanediol di-glycidylether component to produce a coating mixture; applying the coating mixture to at least a portion of one surface of an article; and curing the coating mixture to form a cured coating layer on the article. In some embodiments, the resin is an acrylic resin; and in some embodiments, the acrylic resin is a urethane acrylate. In some embodiments, the reaction initiator is a photoinitiator.

In certain aspects, the disclosure relates to a process for preparing 1,3-propanediol acrylate comprising:
(a) reacting acrylic acid and 1,3-propanediol in a reactor in the presence of an esterification catalyst, with water formed by the reaction being entrained by distillation in a column in the form of a heteroazeotropic mixture with 1,3-propanediol;
(b) condensing the heteroazeotropic mixture of step (a);
(c) separating the condensed heteroazeotropic mixture of step (b) in a decanter thereby yielding an upper organic phase and a lower aqueous phase;
(d) optionally recycling the upper organic phase to the top of the distillation column; and
(e) drawing off the lower aqueous phase.

In certain aspects, the disclosure relates to a process for preparing 1,3-propanediol dimethacrylate comprising transesterifying 1,3-propanediol with an ester of methacrylic acid in the presence of at least one catalyst comprising LiNH2 and LiCl.

In certain aspects, the disclosure relates to a process for preparing 1,3-propanediol diacrylate comprising:
(a) mixing 1,3-propanediol with triethylamine;
(b) adding acryloyl chloride to the mixture of step (a) to produce triethylamine hydrochloride;
(c) filtering the triethylamine hydrochloride from the mixture of step (b) to produce a filtrate without triethylamine hydrochloride;
(d) drying the filtrate of step (c) over anhydrous magnesium sulfate;
(e) distilling the filtrate of step (d) under reduced pressure thereby removing ether and producing a distilled filtrate; and
(f) vacuum distilling the distilled filtrate in the presence of cuprous chloride to produce 1,3-propanediol diacrylate.

In certain aspects, the disclosure relates to a process for preparing 1,3-propanediol diglycidyl ether comprising:
(a) reacting epichlorohydrin and 1,3-propanediol in a reactor in the presence of a catalyst and toluene;
(b) dehydrochlorinating the product of step (a);
(c) filtering the dehydrochlorinated product of step (b);
(d) neutralizing the filtered product of step (c);
(e) distilling the neutralized product of step (d); and
(f) filtering the distilled product of step (e).

### DRAWINGS

FIG. 1 shows a gas chromatography-mass spectrometry (GCMS) analysis of 1,5-pentanediol diacrylate (PDDA) 17.

FIG. 2 shows a GCMS analysis of biologically produced 1,3-propanediol.

FIG. 3 shows a GCMS analysis of 1,5-pentanediol dimethacrylate (PDDMA) 8.

FIG. 4 shows a diagram of a Fourier transform infrared (FTIR) spectrometer.

FIG. 5A shows a destructive-dial depth gauge for measurement on glass. FIG. 5B shows a non-destructive-magnetic/electromagnetic gauge for measurement on steel.

FIG. 6 shows classification of an adhesion cross-cut test. Scale 0-5 (0 is the best), performed on fully dried film (28 days of aging). 1 mm distance for hard surfaces, 2 mm distance for soft surfaces or thickness over 60 μm.

FIG. 7 shows an example of an impact resistance test. FIG. 7A shows a plates side, and FIG. 7B shows a coatings side.

FIG. 8 shows an example of a flexibility/bend test.

FIG. 9 shows an example of an Erichsen-Cupping test. High resistance >8 mm; Low resistance <5 mm.

FIG. 10 shows adhesion of tested coatings to steel via cross-cut ENISO 2409 testing. FIG. 10A shows a steel panel coated with 1,4-butanediol diacrylate. FIG. 10B shows a steel panel coated with biologically-produced propane-1,3-diol diacrylate. FIG. 10C shows a steel panel coated with PDDA 17.

FIG. 11 shows testing results of coatings (Impact Resistance-Falling weight test EN ISO 6272-1, Flexibility-Bend test-ISO 1519, Erichsen Cupping test ISO 1520, Chemical resistance-MEK test, Gloss 20°/60°/80°-Micro TRI gloss (BYK Gardner)-ISO 2813:2014). FIG. 11A shows a steel panel coated with biologically-produced propane-1,3-diol diacrylate and tested for. FIG. 11B shows a steel panel coated with 1,4-butanediol diacrylate. FIG. 11C shows a steel panel coated with PDDA 17.

FIG. 12 shows conversion of double bonds of tested diacrylates. FIG. 12A shows 89.2% conversion of 1,4-butandioldiacrylate. FIG. 12B shows 90.5% conversion of PDDA 17. FIG. 12C shows 88.4% conversion of biologically produced propane-1,3-diol-diacrylate.

FIG. 13 shows a graph of the glass transition temperature of a 1,4-butanediol diacrylate (BDDA) coating.

FIG. 14 shows a graph of the glass transition temperature of a PDDA 17 coating.

FIG. 15 shows a graph of the glass transition temperature of a biologically produced propane-1,3-diol diacrylate coating.

FIG. 16 shows conversion of double bonds of tested dimethacrylates. FIG. 16A shows 79% conversion of 1,3-propanediol dimethacrylate. FIG. 16B shows 76% conversion of 1,4-butanediol dimethacrylate.

FIG. 17 shows a graph of the glass transition temperature of a 1,3-propanedioldimethacrylate coating.

FIG. 18 shows a graph of the glass transition temperature of a 1,4-butanediol dimethacrylate coating.

FIG. 19 shows UV coatings on wood. FIG. 19A shows UV coatings on wood—1st layer. FIG. 19B shows UV coatings on wood—2nd layer—3 acrylates on the left, two methacrylates on the right.

FIG. 20 shows a comparison between baseline composite performance of butanediol diglycidyl ether (BDDGE) and biologically-produced propanediol diglycidyl ether (PDODGE).

### DETAILED DESCRIPTION

One aspect is for a process for preparing 1,3-propanediol acrylate by reacting acrylic acid and 1,3-propanediol in a reactor in the presence of, in some embodiments, an acid as esterification catalyst and, in some embodiments, at least one polymerization inhibitor, the water formed by the reaction being entrained by distillation into a column in the form of a heteroazeotropic mixture with 1,3-propanediol and the heteroazeotropic mixture being subsequently subjected, following condensation, to separation in a decanter to give an upper organic phase which is recycled to the top of the distillation column and a lower aqueous phase which is drawn off.

In the course of this reaction, there can secondary reactions by Michael addition of the 1,3-propanediol onto the double bond of the 1,3-propanediol acrylate.

In some embodiments, the process as defined above is characterized in that the reaction can be conducted with deferred (delayed) introduction of part of the 1,3-propanediol acrylate at the top of the distillation column or into the decanter or into the reactor, the 1,3-propanediol acrylate/acrylic acid molar ratio into the reactor being initially between 0.5 and 1, in some embodiments less than 1, before rising to between 1 and 1.5 following completion of the deferred introduction of the 1,3-propanediol acrylate the difference between the initial and final molar ratios being in some embodiments at least 0.1 and in some embodiments at least 0.2; with an initial temperature (Ti) at the reactor bottom whose lower limit is in some embodiments 70° C. and a final temperature (Tf) at the reactor bottom which is greater than (Ti) and whose upper limit is 110° C.; and under an initial pressure (Pi) of from 3.33×10⁴ Pa (250 mmHg) to 1.33×10⁴ Pa (100 mmHg) and a final pressure (Pf) of from 2.66×10⁴ Pa (200 mmHg) to 0.66×10⁴ Pa (50 mmHg).

In some embodiments, the total mass of 1,3-propanediol is introduced when 85% of the expected mass of water of reaction has been drawn off.

In some embodiments, the deferred introduction of the 1,3-propanediol takes place into the decanter and it is recycled to the top of the distillation column. The upper organic phase of the decanter can be returned to the top of the distillation column by natural overflow.

In some embodiments. the deferred introduction of the 1,3-propanediol takes place directly into the top of the distillation column.

The deferred introduction of the 1,3-propanediol advantageously takes place continuously, it being possible for the rate of deferred introduction of the 1,3-propanediol to be equal to the rate of withdrawal of the water of reaction.

The esterification reaction can be conducted with an initial 1,3-propanediol/acrylic acid molar ratio of 0.92 which rises to 1.12 following completion of the deferred introduction of 1,3-propanediol.

In some embodiments, the reaction is conducted with an initial temperature (Ti) of 80° C. for 30 minutes with regulation of the pressure which varies from 2.933×10⁴ Pa to 2.266×10⁴ Pa and then, while maintaining this pressure, the temperature is allowed to develop until Tf=100° C.

In some embodiments, a distillation column is charged batchwise with acrylic acid, 1,3-propanediol, the stabilizers, and the catalyst (in some embodiments, H₂SO₄) with the flow of light compounds (essentially 1,3-propanediol and 1,3-propanediol acrylate) recovered in the downstream topping columns. The temperature of the reaction medium is between about 70° C. and about 110° C. under a reduced pressure, which is such as to maintain the desired temperature. During the reaction a mixture of 1,3-propanediol, 1,3-propanediol acrylate and water are Teh distilled which separates, following condensation, into two phases in a decanter. The organic phase is returned by overflow to the head of column while the aqueous phase is sent to the feed of the distillation column and/or to the top of a scrubbing column. During the reaction a flow of fresh 1,3-propanediol and/or of light compounds recovered in the topping columns is introduced alternatively into the decanter, at the top of column, or into the reactor (the 1,3-propanediol charging line is used to introduce the deferred 1,3-propanediol).

Following reaction, in some embodiments, the crude reaction product is neutralized with aqueous sodium hydroxide solution in a mixer in order to remove the catalyst and/or the residual acrylic acid. The heterogeneous mixture is separated in the decanter into an aqueous phase, which is sent to the feed of the column, and an organic phase, which constitutes the feed to the bottom of the scrubbing column.

This scrubbing column can be, moreover, supplied at the top with demineralized water and/or with all or part of the water of reaction. At the bottom of column, an alkaline aqueous flow is recovered, which rejoins the feed of column.

At the top of column, the neutralized and washed crude product is sent to the topping column, which allows the excess 1,3-propanediol and some 1,3-propanediol acrylate to be recovered at the top. This mixture can be recycled to the reaction step.

The topped crude product recovered at the bottom of the column feeds a tailing column, which removes the heavy fractions at the bottom, to give the pure ester at the top.

All the aqueous phases of the plant-reaction water, water for neutralizing the crude product, water from the washing column, and other plant waters are sent to column, at the top of which the 1,3-propanediol is principally recovered, and is recycled to the reaction step via a pipe. The waste waters, freed of most of the pollutant organic products, are removed for subsequent biological treatment prior to discharge.

Another aspect is for producing 1,3-propanediol dimethacrylates by reacting 1,3-propanediol with an ester of methacrylic acid.

In some embodiments, the weight ratio of 1,3-propanediol to the ester of methacrylic acid is in the range from about 1:2 to about 1:20, in some embodiments from about 1:5 to about 1:15, and in some embodiments in the range from about 1:6 to about 1:10.

In some embodiments, a combination comprising lithium amide (LiNH₂) and lithium chloride (LiCl) is used for catalyzing the present transesterification. The weight ratio of lithium amide to lithium chloride can, depending on the reaction conditions, be within a wide range. This ratio can be, for example, in the range from about 20:1 to about 1:20, and in some embodiments in the range from about 5:1 to about 1:1.

The amount of catalyst used can be within a wide range. However, processes in which the proportion of catalyst, based on the weight of the 1,3-propanediol used, is in the range from about 0.05 to about 8% by weight, in some embodiments in the range from about 0.01 to about 5% by weight, and in some embodiments in the range from about 0.1 to about 1% by weight, are of particular interest.

The total amount of catalyst used can be added to the reaction mixture at the beginning of the reaction. In some embodiments, part of the catalyst, in some embodiments part of the lithium amide, can be added to the reaction mixture during the course of the reaction. In some embodiments, further catalyst are added to the reaction mixture after a conversion in the range from about 20 to about 80%, in some embodiments in the range from about 30% to about 60%, based on the weight of the 1,3-propanediol used. In particular, processes in which at least about 10% by weight, in some embodiments at least about 20% by weight, of the lithium amide is added to the reaction mixture during the reaction are of interest.

The reaction can be carried out under superatmospheric or subatmospheric pressure. In some embodiments, the transesterification can be carried out at a pressure in the range from about 200 to about 2000 mbar, in some embodiments in the range from about 500 to about 1300 mbar.

The reaction temperature can, especially as a function of the pressure, likewise be within a wide range. In some embodiments, the reaction is carried out at a temperature in the range from about 60° C. to about 150° C., in some embodiments in the range from about 70° C. to about 140° C., and in some embodiments from about 90° C. to about 130° C.

In some embodiments, the temperature at which the reaction occurs is increased during the course of the reaction. The temperature at the beginning of the reaction, in particular up to a conversion of about 80%, in some embodiments up to a conversion of about 70%, based on the weight of the 1,3-propanediol used, can be in the range from about 90° C. to about 110° C. and that towards the end of the reaction, in particular after a conversion of about 80%, in some embodiments after a conversion of about 90%, based on the weight of the 1,3-propanediol used, can be in the range from about 115° C. to about 130° C.

The transesterification can be carried out either continuously or batchwise. The process can be carried out in bulk, i.e. without use of a further solvent. If desired, an inert solvent can also be used. Such solvents include, for example, benzene, toluene, n-hexane, cyclohexane and methyl isobutyl ketone (MIBK), and methyl ethyl ketone (MEK).

In some embodiments, all components, for example the 1,3-propanediol, the methacrylic ester, and the catalyst, are mixed, after which this reaction mixture is heated to boiling. The alcohol liberated, for example methanol or ethanol, can subsequently be removed from the reaction mixture by distillation, if appropriate as an azeotrope with methyl methacrylate or ethyl methacrylate.

The reaction times depend, for example, on the parameters selected, for example pressure and temperature. However, they are generally in the range from about 1 to about 24 hours, in some embodiments from about 5 to about 20 hours, and in some embodiments from about 6 to about 12 hours. In the case of continuous processes, the residence times are generally in the range from about 0.5 to about 24 hours, in some embodiments from about 1 to about 12 hours, and in some embodiments from about 2 to about 3 hours.

The reaction can take place with stirring, with the stirring rate in some embodiments being in the range from about 50 to about 2000 rpm, and in some embodiments in the range from about 100 to about 500 rpm.

The pH can be within a wide range. The reaction can be carried out, e.g., at a pH in the range from about 8 to 1 about 4, in some embodiments from about 9 to about 13.

To prevent undesirable polymerization of the methacrylates, polymerization inhibitors can be used in the reaction. These compounds, for example hydroquinones, hydroquinone ethers such as hydroquinone monomethyl ether or di-tert-butylcatechol, phenothiazine, N,N′-(diphenyl)-p-phenylenediamine, 4-hydroxy-2,2,6,6-tetramethylpiperidin-1-oxyl, p-phenylenediamine, methylene blue or sterically hindered phenols, are widely known in the art. These compounds can be used individually or in the form of mixtures and are generally commercially available. The mode of action of the stabilizers is usually that they act as free-radical scavengers for the free radicals occurring in the polymerization. Further details may be found in the relevant specialist literature, in particular Römpp-Lexikon Chemie; editor: J. Falbe, M. Regitz; Stuttgart, New York; 10th Edition (1996).

In some embodiments, amines are used as polymerization inhibitor, in some embodiments N,N′-(diphenyl)-p-phenylenediamine is used. Based on the weight of the total reaction mixture, the proportion of inhibitors, either individually or as a mixture, can generally be about 0.01 to about 0.5% (wt/wt).

These polymerization inhibitors can be added to the reaction mixture before or at the beginning of the reaction. Furthermore, small proportions of the polymerization inhibitors employed can be introduced during the transesterification. Processes in which part of the polymerization inhibitor is added via the column runback can be used. In some embodiments, mixtures containing methyl methacrylate, hydroquinone monomethyl ether and 4-hydroxy-2,2,6,6-tetramethylpiperidin-1-oxyl are used. This mixture makes it possible, in particular, to avoid undesirable polymerization within the distillation column.

Furthermore, oxygen can be used for the inhibition. This can be used, for example, in the form of air, with the amounts introduced being such that the content in the gas phase above the reaction mixture remains below the explosive limit. Amounts of air in the range from about 0.05 to about 0.5 l per hour and mole of 1,3-propanediol can be used. In batch processes, this amount can be based on the amount of 1,3-propanediol originally used. In the case of continuous processes, this amount can be based on the amount of 1,3-propanediol fed in. It is likewise possible to use inert gas/oxygen mixtures, e.g. nitrogen/oxygen or argon/oxygen mixtures.

In some embodiments, a combination of oxygen with at least one amine, in some embodiments N,N′-(diphenyl)-p-phenylenediamine, can be used for inhibition.

In some embodiments, the alcohol liberated from the methacrylate used, for example methanol and/or ethanol, can be separated off by distillation. Here, a mixture containing, for example, methyl methacrylate and methanol can advantageously be separated off. Part of the mixture which has been separated off can be recirculated to the next batch. In this modification, the proportion which can be recirculated of the mixture which has been separated off can be obtained at the end of the reaction, in some embodiments after a conversion of about 80%, in some embodiments after a conversion of about 90%, of the 1,3-propanediol used. For example, the proportion of the recirculated mixture at the beginning of the next batch can be in the range from about 10 to about 50%, based on the total weight of methacrylic ester to be transesterified.

Batch processes in which methyl methacrylate is added during the transesterification can be used. In some embodiments, methyl methacrylate is removed together with methanol from the reaction mixture. The weight ratio of the amount of methyl methacrylate added during the transesterification to the amount of methanol/methyl methacrylate mixture separated off can be in the range from about 2:1 to about 1:2.

In the case of batch processes, excess starting material, in particular the unreacted ester of methacrylic acid, can be separated off by distillation towards the end of the reaction. This too can be reused without further purification in the next batch.

The methanol-or ethanol-rich distillate obtained at the beginning of the reaction can likewise be recycled, for example by introduction into a coupled plant for preparing the methacrylate ester to be transesterified.

A suitable plant for carrying out the present transesterification can comprise, for example, a stirred tank reactor provided with agitator, steam heating, distillation column and condenser. Such plants are known per se and are described, for example, in Ullmanns Encyclopedia of Industrial Chemistry (6th Edition), Verlag Wiley-VCH, Weinheim 2003, Volume 10, page 647. The size of the plant depends on the amount of 1,3-propanediol dimethacrylate to be prepared, with the present process being able to be carried out either on a laboratory scale or on an industrial scale. According to in some embodiments, the stirred tank reactor can accordingly have a tank volume in the range from about 1 m³ to about 30 m³, in some embodiments from about 3 m³ to about 20 m³. The agitator of the reactor tank can, in particular, be configured in the form of an anchor stirrer, impeller, paddle stirrer, or Inter-MIG stirrer.

The task of the distillation column can be to ensure that a methanol-or ethanol-rich azeotrope is taken off in order to minimize the losses of starting ester which is inevitably discharged. The distillation column can have one, two, or more separation stages. The number of separation stages is the number of trays in the case of a tray column or the number of theoretical plates in the case of a column containing ordered packing or random packing elements. Examples of trays in a multistage distillation column are bubblecap trays, sieve trays, tunnel trays, valve trays, slotted trays, sieve-slotted trays, sieve-bubblecap trays, nozzle trays, centrifugal trays; examples of random packing elements in a multistage distillation column are Raschig rings, Lessing rings, Pall rings, Berl saddles, Intalox saddles; and examples of ordered packing in a multistage distillation column are Mellapak (Sulzer), Rombopak (Kühni), Montz-Pak (Montz). Conversion-dependent adaptation of the reflux ratio enables, for example when using methyl methacrylate, a proportion of methanol in the distillate which is above about 60% to be obtained over a wide conversion range.

Suitable condensers which can be present in the plant for carrying out the present transesterification include, e.g., plate heat exchangers and shell-and-tube heat exchangers.

To increase the quality further of the 1,3-propanediol dimethacrylate and, in particular, to separate off the catalyst, the mixture obtained can be purified by known methods.

In some embodiments, the product mixture obtained can be purified by a filtration process (see, e.g., W. Gosele, Chr. Alt in Ullmann's Encyclopedia of Industrial Chemistry, (6th Edition), Verlag Wiley-VCH, Weinheim 2003, Volume 13, pages 731 and 746), which can be carried out using customary filtration aids such as aluminum silicate (Perlite). For example, it is possible to use, e.g., continuously operable filters for a washcoat filtration or candle filters.

In some embodiments, a filtrate product can be further distilled. Owing to the tendency of the monomer to polymerize, distillation processes in which the thermal stress on the substance to be distilled is minimized are advisable. Apparatuses in which the monomer is continuously vaporized from a thin layer, e.g. falling film evaporators and evaporators having a rotating wiper system, are well suited. Short path evaporators can also be used. Such apparatuses are known (Ullmanns Encyclopedia of Industrial Chemistry (6th Edition), Verlag Wiley-VCH, Weinheim 2003, Volume 36, page 505). Thus, for example, a continuous evaporator having a rotating wiper system and a superposed column can be used. The distillation can, for example, be carried out at a pressure in the range from about 1 to about 40 mbar and an evaporator temperature of from about 120° C. to about 150° C.

A further aspect is for the production of 1,3-propanediol diacrylate by dehydrochlorination between acryloyl or methacryloyl chloride and 1,3-propanediol.

1,3-propanediol and an approximately equimolar amount of a tertiary amine such as pyridine or triethylamine are poured into a solvent, in some embodiments selected from aromatic hydrocarbons such as benzene, toluene, and xylene; aliphatic or alicyclic hydrocarbons such as hexane and cyclohexane; or ether, in a reactor equipped with stirring blades, into which an approximately equimolar amount of acryloyl or methacryloyl chloride is added dropwise gradually under stirring at, in some embodiments about 0° C. to about 10° C. The mixture is reacted sufficiently for about 5 to about 8 hours after dropping, and the formed ammonium hydrochloride is removed from the reaction mixture by filtering.

Next, the solvent is removed by, e.g., reduced pressure distillation, and the residue is thereafter vacuum distilled to give 1,3-propanediol diacrylate as the end product, as a colorless and transparent liquid.

In some embodiments, approximately one mol of 1,3-propanediol and approximately two mol of a tertiary amine such as pyridine or triethylamine are poured into a solvent, in some embodiments selected from aromatic hydrocarbons such as benzene, toluene, and xylene; aliphatic or alicyclic hydrocarbons such as hexane and cyclohexane; or ether, in a reactor equipped with stirring blades, into which approximately two mols of acyloyl or methacryloyl chloride is added dropwise gradually under stirring at about 0° C. to about 10°. The mixture is reacted sufficiently for about 5 to about 8 hours after dropping, and the formed ammonium hydrochloride is removed from the reaction mixture by filtering. Next, the solvent is removed by, e.g., reduced pressure distillation, and the residue is thereafter vacuum distilled to give 1,3-propanediol diacrylate as the end product of colorless, transparent liquid.

The resulting diacrylate of 1,3-propanediol has a methyl group as a side chain in the glycol component, and therefore possesses several characteristics, that is, a polarity effect due to an electron-releasing property of the methyl group, elimination of a methyne hydrogen atom bound by a covalent bond to the same carbon atom to which the methyl group is bound and high reactivities of a terminal polymerizable double bond and a terminal hydroxyl group. It may be therefore expected to be used in broad applications such as starting materials or intermediates for thermosetting paints, adhesives and cross-linking agents and then copolymer modifiers.

An additional aspect is for production of 1,3-propanediol diglycidyl ether.

In some embodiments, 1,3-propanediol and a catalyst are added into a reactor, stirred evenly, followed by addition of epichlorohydrin dropwise at about 60° C. to about 80° C. Dropwise time is about 1 to about 3 hours, keeping for about 1 to about 2 hours. The mixture is then stirred, and an amount of solvent, in some embodiments, toluene are added to the reactor, and then an alkali is added at a temperature in a range of about 35° C. to about 45° C., for about 2 to about 4 hours, and after that, kept for about 1 to about 2 hours.

After the reaction, the product is transferred to a funnel, in some embodiments a Buchner funnel, for suction filtration. The filtrate is neutralized by adding a certain amount of acid, in some embodiments phosphoric acid, and then transferred to a separatory funnel for liquid separation. After liquid separation, the solvent is removed to obtain 1,3-propanediol diglycidyl ether.

In some embodiments, production of 1,3-propanediol diglycidyl ether can be performed by mixing 1,3-propanediol and catalyst in a reactor, stirring evenly, and adding epichlorohydrin dropwise at about 60° C. to about 80° C., dropwise time is about 3 hours, kept for about 1 hour. Next, the mixture is stirred, and an amount of solvent, in some embodiments toluene, is added into the reactor, then alkali is added at about 30° C. to about 45° C., and kept for about 1 hour after it is finished.

After the reaction, the product is transferred to a funnel, in some embodiments a Buchner funnel, for suction filtration. The filtrate is neutralized by adding a certain amount of acid, in some embodiments phosphoric acid, and then transferred to a separatory funnel for liquid separation. After liquid separation, the solvent was removed to obtain 1,3-propanediol diglycidyl ether.

In some embodiments, in the above-mentioned methods for preparing 1,3-propanediol diglycidyl ether, the catalyst is boron trifluoride-diethyl ether or perchloric acid. In some embodiments, the mass ratio of the catalyst to 1,3-propanediol is about 0.5% to about 1%. In some embodiments, the mass ratio of catalyst to 1,3-propanediol is about 0.7%.

In some embodiments, the molar mass ratio of epichlorohydrin to 1,3-propanediol is about 2.2:1 to about 2.4:1; in some embodiments about 2.2:1, about 2.3:1, or about 2.4:1.

In some embodiments, the reaction temperature for the first step of the reaction is about 60° C. to about 80° C.; in some embodiments about 60° C., about 70° C., or about 80° C.

In some embodiments, the mass ratio of solvent to 1,3-propanediol is about 2.5:1 to about 4:1; in some embodiments about 3.5:1.

In some embodiments, the reaction temperature for the second step of the reaction is about 35° C. to about 45° C.; in some embodiments about 35° C., about 40° C., or about 45° C.

In some embodiments, NaOH solution with a mass concentration of about 32%, about 48%, or solid NaOH is added in about 4 hours.

In some embodiments, the molar mass ratio of NaOH to epichlorohydrin is about 1:1, about 1.05:1, or about 1.1:1.

Compositions comprising 1,3-propanediol acrylates, monoacrylates, di(meth)acrylates, and/or diglycidyl ethers.

The 1,3-propanediol acrylates, monoacrylates, di(meth)acrylates, and/or diglycidyl ethers produced by a process described herein can be used in variety of compositions. For example, one aspect is for an adhesive or sealant comprising a 1,3-propanediol diacrylate component, 1,3-propanediol monoacrylate component, 1,3-propanediol dimethacrylate component, and/or a 1,3-propanediol di-glycidylether component. Another aspect is for an ink comprising a 1,3-propanediol diacrylate component, and/or 1,3-propanediol monoacrylate component; and in some embodiments, the ink is a flexographic ink, an inkjet ink, or a screen ink. A further aspect is for a coating comprising a 1,3-propanediol diacrylate component, and/or 1,3-propanediol monoacrylate component; and in some embodiments, the coating is a metal coating or a wood coating. An additional aspect is for an artificial marble comprising a 1,3-propanediol dimethacrylate component. A further aspect is for a coating comprising a 1,3-propanediol dimethacrylate component and/or a 1,3-propanediol di-glycidylether component; in some embodiments, the coating is a conformal coating or a wood coating; and in some embodiments, the coating comprises a 1,3-propanediol di-glycidylether component and is a protective coating, a marine coating, or an electronics coating. An additional aspect is for an electrical potting composition comprising a 1,3-propanediol dimethacrylate component. Another aspect is for an encapsulation composition comprising a 1,3-propanediol dimethacrylate component; and in some embodiments, the encapsulation composition is a flooring and/or waterproofing encapsulation composition. A further aspect is for a composite comprising a 1,3-propanediol di-glycidylether component.

Compositions containing polymers of 1,3-propanediol acrylates, di(meth)acrylates, and/or diglycidyl ethers containing, in some embodiments, from about 10 to about 95 weight percent, in some embodiments, from about 25 to about 75 weight percent, or in some embodiments, from about 30 to about 60 weight percent, of a 1,3-propanediol acrylate, di(meth)acrylate, and/or diglycidyl ether component. Polymers of 1,3-propanediol acrylates, di(meth)acrylates, and/or diglycidyl ethers (e.g., homopolymers or copolymers) may be prepared by any standard polymerization process including free radial, anionic, etc.

The catalyst employed can be a free radical initiator or a redox catalyst. Exemplary catalysts which can be employed include, but are not limited to, free radicals initiators such as hydrogen peroxide, peracetic acid, t-butyl hydroperoxide, di-t-butyl peroxide, dibenzoyl peroxide, benzoyl hydroperoxide, 2,4-dichlorobenzoyl peroxide, 2,5-dimethyl-2,5-bis (hydroperoxy) hexane, perbenzoic acid, t-butyl peroxypivalate, t-butyl peracetate, azo-bis-iso-butyronitrile, ammonium persulfate, potassium persulfate, sodium perphosphate, potassium perphosphate, isopropyl peroxycarbonate, 2,2′-azobis (2,4-dimethylvaleronitrile) etc.; and redox catalyst systems such as sodium persulfate-sodium formaldehyde sulfoxylate, cumene hydroperoxide-sodium metabisulfite, hydrogen peroxide-ascorbic acid, sulfur dioxide-ammonium persulphate, etc. The catalysts are employed in the usual catalytically effective concentrations which are known to those skilled in the art of emulsion polymerization.

The polymerization can be carried out in the presence of an organic solvent which will not interfere with the polymerization reaction. Illustrative of the solvents which may be employed are ethoxyethyl acetate, methylene chloride, ethanol, iso-propanol, n-propanol, n-butanol, iso-butanol, tertbutanol; the methyl, ethyl, propyl or butyl esters of acetic acid, acetone, methyl ethyl ketone, benzene, toluene and the like.

The reaction may be carried out at a temperature of from about 50° C. to about 160° C., and in some embodiments of from about 90° C. to about 130° C. The reaction may be performed as sub-or superatmospheric pressures.

Reaction time is not critical and may vary from less than several hours to several days or more depending upon the reaction batch size, pressure, temperature, etc. selected.

The crosslinkers which may be employed will vary with the type of functional crosslinking sites on the copolymer employed. Thus, for copolymers containing active hydroxyl groups crosslinkers including polyepoxides (such as cycloaliphatic epoxides and diglycidyl epoxides), polyfunctional isocyanates, etherated amino-formaldehyde resins, etc. and mixtures thereof may be employed. For copolymers containing active carboxyl groups crosslinkers including polyepoxides (such as cycloaliphatic epoxides and diglycidyl epoxides), aziridines, carbodiimides, etherated amino-formaldehyde resins, etc. and mixtures thereof may be employed. For copolymers containing active amide groups, crosslinkers including polyfunctional isocyanates, polyepoxides (such as cycloaliphatic epoxides and diglycidyl epoxides), etherated amino-formaldehyde resins, etc. and mixtures thereof may be employed. For copolymers which contain mixtures of active hydroxyl, carboxyl and/or amide groups, mixtures of suitable crosslinkers may be utilized.

In general, the amount of crosslinker which should be employed will be dependent on the equivalent weight of the crosslinker used, the inherent flexibility of the crosslinker used, the reactivity of the crosslinker with itself, and the degree of hardness/softness or flexibility desired in the final coating. These amounts will vary with particular functional monomer and crosslinker combination selected and will be known to one skilled in the art.

The catalyst employed in the crosslinking reaction, if indeed a catalyst is required for the given active site/crosslinker combination, will vary in accordance with the particular active site/crosslinker combination selected. The amounts and types of catalysts necessary will be well known to one skilled in the art. Thus, for example, with aminoplast crosslinkers catalysts such as p-toluene sulfuric acid, naphthalene sulfonic acid, phosphoric acid, dinonyl naphthalene disulfonic acid, or the stannous salt of trifluoromethane sulfuric can be used. With cycloaliphatic epoxides catalysts such as stannous octanoate, dibutyltin dilaurate, triflic acid, the reaction product of triflic acid and stannous oxide, diethyl ammonium triflate, etc. can be used.

In some embodiments, a composition (e.g., an adhesive, sealant, flexographic ink, inkjet ink, screen ink, metal coating, or wood coating) comprising 1,3-propanediol diacrylate, and/or 1,3-propanediol monoacrylate has one or more properties selected from the group consisting of a harder coating, improved mechanical properties, lower flexibility, a harder film, higher tensile strength, lower viscosity, and increased chemical resistance relative to a composition comprising 1,4-Butanediol (BDO) diacrylate, 1,6-Hexanediol (HDO) diacrylate, Pentanediol diacrylate and/or other diacrylates containing diols other than 1,3-propanediol.

In some embodiments, a composition (e.g., an adhesive, sealant, artificial marble, composite, conformal coating, electrical potting composition, encapsulation composition, industrial flooring, wood coating or waterproofing composition) comprising 1,3-propanediol dimethacrylate has one or more properties selected from the group consisting of a harder coating, improved mechanical properties, lower flexibility, a harder film, higher tensile strength, lower viscosity and increased chemical resistance relative to a composition comprising 1,4-Butanediol (BDO) dimethacrylate, 1,6-Hexanediol (HDO) dimethacrylate, Pentanediol dimethacrylate, and/or other dimethacrylates containing diols other than 1,3-propanediol.

In some embodiments, a composition (e.g., an adhesive, sealant, composite material, or coating such as an industrial coating, wood coating, metal coating, marine coating, or electronic coating) comprising 1,3-propanediol di-glycidylether has one or more properties selected from the group consisting of lower viscosity, higher reactivity, harder film, higher tensile strength and increased chemical resistance relative to a composition comprising 1,4-Butanediol (BDO) di-glycidylether, 1,6-Hexanediol (HDO) di-glycidylether, Pentanediol di-glycidylether, and/or other di-glycidylethers containing diols other than 1,3-propanediol.

Coating compositions disclosed herein comprise a reactive diluent, a resin, and a reaction initiator, with the reactive diluent comprising a 1,3-propanediol diacrylate component, 1,3-propanediol monoacrylate component, and/or a 1,3-propanediol di-glycidylether component described above.

The resin component can comprise an acrylic resin, phenolic resin, nitrile resin, ethylene vinyl acetate resin, polyurethane resin, polyurea or urea-formaldehyde resin, isocyanate resin, styrene-butadiene resin, styrene-acrylic resins, vinyl acrylic resin, aminoplast resin, melamine resin, polyisoprene resin, epoxy resin, ethylenically unsaturated resin, bismaleimide binders, vinyl ether resins, beta unsaturated carbonyl groups, acrylate resins, acrylated isocyanurate resins, isocyanurate resins, acrylated urethane resins, acrylated epoxy resins, alkyd resins, hide glue, varnish, and radiation cured crosslinked acrylate binders, nitrile rubbers, epoxies, surfactants, polyethylene glycol, polyvinylpyrrolidones, polylactic acid (PLA), polyvinylpyrrolidone/vinyl acetate copolymers, polyvinyl alcohols, carboxymethyl celluloses, hydroxypropyl cellulose starches, polyethylene oxides, polyacrylamides, polyacrylic acids, cellulose ether polymers, polyethyl oxazolines, esters of polyethylene oxide, and polypropylene oxide copolymers, urethanes of polyethylene oxide, and any mixtures thereof. In some embodiments, the resin is an acrylic resin chosen from urethane acrylate resins, urethane methacrylate resins, epoxy acrylate resins, and mixtures thereof, and in some embodiments, difunctional epoxy acrylate resins, aliphatic urethane acrylate resins, hydrophobic urethane acrylate resins, aromatic urethane acrylate resins, polyether urethane acrylate resins, aliphatic urethane methacrylate resins, hydrophobic urethane methacrylate resins, aromatic urethane methacrylate resins, polyether urethane methacrylate resins, and mixtures thereof. In some embodiments, the total amount of resin present will typically be from about 5 wt % to about 60 wt %, from about 10 wt % to about 55 wt %, from about 15 wt % to about 50 wt %, from about 20 wt % to about 45wt %, from about 25 wt % to about 40 wt %, from about 30 wt % to about 45 wt % based on the total weight of the composition. In some embodiments, the total amount of resin present is about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 wt % or more based on the total weight of the composition.

In some embodiment, the reaction initiator can be a photoinitiator or a thermal initiator.

A photoinitiator compound is defined in the present invention as a substance that may generate start radicals for the radical polymerization reaction of ethylenic double bonds, such as for vinyl ethers, (meth)acrylates or itaconates, when the substance is exposed to UV irradiation or to visible light. There are three classes of photoinitiators known for free radical polymerization reactions, namely type I photoinitiators, type II photoinitiators and co-initiators. The first class are type I photoinitiators, which are substances that undergo a fragmentation forming two radicals, when they are in the excited state. At least one of the radicals react as start radical for the radical polymerization reaction of ethylenic groups. For the class of type Il photoinitiators a co-initiator (which is the third class of photoinitiator) is required to create the start radical. In the primary process after the photon absorption the photoinitiator abstracts a hydrogen atom from a co-initiator to produce a rather unreactive ketyl radical. Only the radical being created by the H atom abstraction from the co-initiator can react as start radical for the radical polymerization reaction. Typically type II photoinitiators are those of the group of benzophenones, thioxanthones, fluorenones, xanthones and anthraquinones. Co-initiators are typically tertiary amines, or more specifically aromatic tertiary amines, such as dimethylamino benzoates or derivatives thereof.

In some embodiments, the photoinitiator is 2,4,6-trimethylbenzoyl-phenylphosphinate oxide (TPO-L), camphorquinone, 4,4′-bis(diethylamino)benzophenone, 4,4′-bis (diethylamino) benzophenone combined with N-phenylglycine (NPG), with ethyl-4-(dimethylamino)benzoate (EDB), with N-diisopropylethylamine (DIPEAN) or with 4-(dimethylamino)benzonitrile (DMABN), biacylphosphine oxide (BAPO), titanium bis (.eta.5-2,4-cylcopentadien-1-yl)-bis (2,6-difluoro-3-(1H-pyrrol-1-yl)-phenyl) (Irgacure 784), 1-[4-(2-hydroxyethoxy)-phenyl]-2-hydroxy-2-methyl-1-propane-1-one (Irgacure 2959), 2,4,6-trimethylbenzoyldiphenylphosphine oxide (TPO), 2,2-dimethoxyphenyl-2-acetophenone (DMPA), or a mixtures thereof.

In some embodiments, the total amount of reaction initiator present will typically be from about 0.1 wt % to about 10 wt %, from about 0.2 wt % to about 9 wt %, from about 0.3 wt % to about 8 wt %, from about 0.4 wt % to about 7 wt %, from about 0.5 wt % to about 6 wt %, from about 1 wt % to about 5 wt %, from about 2 wt % to about 4 wt %, based on the total weight of the composition. In some embodiments, the total amount of reaction initiator present is about 0.1, 0.2, 0.3, 0.4, 0.5, 0.6, 0.7, 0.8, 0.9, 1, 2, 3, 4, 5, 6, 7, 8, 9, 10, or more based on the total weight of the composition.

In some embodiments, the total amount of reactive diluent present will typically be from about 5 wt % to about 60 wt %, from about 10 wt % to about 55 wt %, from about 15 wt % to about 50 wt %, from about 20 wt % to about 45 wt %, from about 25 wt % to about 40 wt %, from about 30 wt % to about 45 wt % based on the total weight of the composition. In some embodiments, the total amount of reactive diluent present is about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 wt % or more based on the total weight of the composition.

In some embodiments, a coating composition comprises a crosslinkable component and a crosslinking component.

As used herein, the term “crosslinkable component” means a component used to provide a coating composition wherein the crosslinkable component is inclusive of a compound, polymer, copolymer or a polydisperse mixture of compounds, polymers and/or copolymers all having nucleophilic or active-hydrogen-containing functional groups that are capable of reacting with functional groups on the crosslinking component during the curing step to produce a coating in the form of crosslinked structures.

As used herein, the term “crosslinking component” shall mean a component used to provide a coating composition wherein the crosslinking component is inclusive of a compound, polymer, copolymer or a polydisperse mixture of compounds, polymers, and/or copolymers all having functional groups that are capable reacting with the functional groups on the crosslinkable component during the curing step to produce a coating in the form of crosslinked structures. Crosslinking reactions are known and understood by those of ordinary skill in the art.

The crosslinkable polymer can comprise monomeric units such as, for example, a 1,3-propanediol diacrylate component, and/or 1,3-propanediol monoacrylate component as disclosed herein. In some embodiments, the crosslinkable polymer can further comprise: styrene; alkyl styrene; vinyl toluene; and alkyl acrylates or alkyl methacrylates having alkyl groups of from 1-18 carbon atoms. Suitable alkyl acrylates and alkyl methacrylates include, for example: methyl acrylate; methyl methacrylate; ethyl acrylate; ethyl methacrylate; isopropyl acrylate; isopropyl methacrylate; n-butyl acrylate; n-butyl methacrylate; isobutyl acrylate; isobutyl methacrylate; pentyl acrylate; pentyl methacrylate; hexyl acrylate; hexyl methacrylate; 2-ethyl hexyl acrylate; 2-ethyl hexyl methacrylate; nonyl acrylate; nonyl methacrylate; lauryl acrylate; lauryl methacrylate; stearyl acrylate; and stearyl methacrylate. Suitable acrylates and methacrylates can also include cycloaliphatic acrylate and cycloaliphatic methacrylates such as, for example: cyclohexyl acrylate; cyclohexyl methacrylate; isobornyl acrylate; isobornyl methacrylate; trimethylcyclohexyl acrylate; trimethylcyclohexyl methacrylate; glycidyl acrylate; glycidyl methacrylate; benzyl acrylate; and benzyl methacrylate. Combinations of any of the above can also be suitable.

The crosslinkable component comprises an acrylic polymer that can be linear, branched, or the crosslinkable component can have a combination of both linear and branched polymers.

Linear and branched acrylic polymers, in some embodiments, have a weight average molecular weight (Mw) varying in the range of from about 1,000 to about 40,000; in some embodiments, varying in the range of from about 1,500 to about 30,000; and in some embodiments, varying in the range of from about 2,000 to about 25,000. Suitable acrylic polymers have a Tg varying in the range of from of about −20° C. to about 100° C.; in some embodiments, varying in the range of from about 0° C. to about 90° C.; and in some embodiments, varying in the range of from about 40° C. to about 80° C.

Polymers and copolymers can be obtained by conventional processes well known to those of ordinary skill in the art. Typically, solvent is added into a reactor and brought to reflux temperature under a nitrogen blanket. Upon attaining the desired polymerization temperature, an initiator and the monomer mixture are fed to the reactor over a period of time. The monomer mixture and initiator can be added into the reactor all at once or fed in portions into the reactor.

Conventional initiators known to those of ordinary skill in the art can be used herein alone or in combination with other initiators. Such conventional initiators include, without limitation: azo initiators such as, for example, 2,2′-azobis (2,4dimethylpentane nitrile); peroxides such as for example, di-tertiarybutyl peroxide; and hydroperoxides. Commercially available peroxy type initiator t-butylperacetate or TRIGANOX® B from Akzo Nobel is also suitable for use herein.

A branched acrylic polymer can be produced by a polymerization process that is described in U.S. Pat. Nos. 4,680,352 and 5,290,633, which are incorporated herein by reference. In this process, the branched polymers are made in two stages. In the first stage, macromonomers, using conventional cobalt (II) or (III) chelate chain transfer agent, are produced to ensure that the macromonomer is provided with one terminal ethylenically unsaturated group that can be further polymerized. During the second stage, the monomer mixture described earlier is added to the reactor containing the macromonomers. The monomers polymerize with the ethylenically unsaturated group on the macromonomer to produce the branched acrylic polymer. In a branched acrylic polymer, the desired secondary amine, primary hydroxyl and secondary hydroxyl group monomers can be added to either the macromonomer or the backbone.

The polymer can also be a core-shell polymer. The core-shell polymer has a solvent insoluble core, and a solvent soluble shell that is chemically attached to the core. In some embodiments, the shell is in the form of macromonomer chains or arms attached to the core. The core-shell polymer is a polymer particle dispersed in organic media, wherein the polymer particle is stabilized by what is known as steric stabilization. The average particle size of the core ranges from about 0.1 μm to about 1.0 μm; in some embodiments, from about 0.15 μm to about 0.6 μm; and in some embodiments, from about 0.15 μm to about 0.6 μm.

A core-shell polymer includes in the range of from about 10% to about 90%, in some embodiments, in the range of from about 50% to about 80% all in weight percent based on the weight of the core-shell polymer, of a core formed from high molecular weight polymer having a weight average molecular weight of about 50,000 to about 500,000; in some embodiments, in the range of from about 50,000 to about 200,000; in some embodiments, more in the range of from about 50,000 to about 150,000. The arms make up about 10% to about 90%; in some embodiments, about 20% to about 50%, all in weight percent based on the weight of the core-shell polymer. The arms are formed from a relatively lower molecular weight polymer having weight average molecular weight in the range of from about 1,000 to about 50,000; in some embodiments, in the range of from about 2,000 to about 40,000; and in some embodiments, in the range of from about 3,000 to about 30,000.

The core of the dispersed core-shell polymer is comprised of one or more polymerized acrylic monomers. Suitable monomers include, for example, a 1,3-propanediol diacrylate component, and/or 1,3-propanediol monoacrylate component as disclosed herein. In some embodiments, the suitable monomers can further comprise: styrene, alkyl acrylates, and alkyl methacrylates having alkyl carbon atoms in the range of from 1 to 18, in some embodiments, in the range of from 1 to 12; ethylenically unsaturated monocarboxylic acid, such as, for example, acrylic acid and methacrylic acid; silane-containing monomers, and epoxy containing monomers, such as glycidyl (meth)acrylate. The core can be crosslinked using optional monomers, for example, amine containing monomers, hydroxyalkyl acrylates, hydroxyalkyl methacrylates or acrylonitrile. Optionally, the core may be crosslinked through the use of diacrylates or dimethacrylates, such as, allyl methacrylate or through post reaction of hydroxyl moieties with polyfunctional isocyanates or carboxylic moieties with epoxy moieties.

The core or the macromonomer arms attached to the core are polymerized from monomers having secondary amine monomers, primary hydroxyl monomers, and secondary hydroxyl monomers. In addition, the arms can also contain polymerized monomers, such as, for example, styrene and alkyl acrylates and alkyl methacrylates wherein the alkyl portion has 1 to 18 carbon atoms.

The process for making the core-shell polymer is described in U.S. Pat. No. 5,659,136, which is incorporated herein by reference.

The coating composition may also comprise, as the crosslinkable component, one or more polyester resins as known in the art. Suitable polyesters have at least one or more functionalities that are reactive with isocyanate, for example, hydroxyl groups. A suitable polyester resin has a weight average molecular weight (Mw) within the range of from about 2,000 to about 20,000, in some embodiments, within the range of from about 3,000 to about 10,000. A suitable polyester resin has a Tg within the range of from about 20° C. to about 100° C.; in some embodiments, within in the range of from about 0° C. to about 90° C.; and in some embodiments, within the range of from about 20° C. to about 80° C.

Polyesters suitable for use can be polyesters known conventionally or used commercially by those of ordinary skill in the coating art. Suitable polyesters can be polymerized from suitable polyacids, including cycloaliphatic polycarboxylic acids, and suitable polyols, which include polyhydric alcohols. Examples of suitable cycloaliphatic polycarboxylic acids are tetrahydrophthalic acid, hexahydrophthalic acid, 1,2-cyclohexanedicarboxylic acid, 1,3-cyclohexanedicarboxylic acid, 1,4-cyclohexanedicarboxylic acid, 4-methylhexahydrophthalic acid, endomethylenetetrahydrophthalic acid, tricyclodecanedicarboxylic acid, endoethylenehexahydrophthalic acid, camphoric acid, cyclohexanetetracarboxylic acid and cyclobutanetetracarboxylic acid. The cycloaliphatic polycarboxylic acids can be used not only in their cis but also in their trans form or a mixture thereof. Examples of suitable polycarboxylic acids, which, if desired, can be used together with the cycloaliphatic polycarboxylic acids, are aromatic and aliphatic polycarboxylic acids, such as, for example, phthalic acid, isophthalic acid, terephthalic acid, halogenophthalic acids, such as, tetrachloro-or tetrabromophthalic acid, adipic acid, glutaric acid, azelaic acid, sebacic acid, fumaric acid, maleic acid, trimellitic acid, and pyromellitic acid.

Suitable polyhydric alcohols include ethylene glycol, propanediols, butanediols, hexanediols, neopentylglycol, diethylene glycol, cyclohexanediol, cyclohexanedimethanol, trimethylpentanediol, ethylbutylpropanediol, ditrimethylolpropane, trimethylolethane, trimethylolpropane, glycerol, pentaerythritol, dipentaerythritol, tris (hydroxyethyl) isocyanate, polyethylene glycol and polypropylene glycol. If desired, monohydric alcohols, such as, for example, butanol, octanol, lauryl alcohol, ethoxylated or propoxylated phenols may also be included along with polyhydric alcohols. The details of polyester suitable for use are further provided in the U.S. Pat. No. 5,326,820, which is incorporated herein by reference.

The crosslinkable component may also include various polyether polyols to improve the properties of the coating. Various polyether polyols may be used as is known to those of ordinary skill in the art. In one embodiment, polyether polyol is polytrimethylene ether polyol. The polytrimethylene ether polyol has a number average molecular weight in the range of from 500 to 5,000 and is present in the crosslinkable component in the range of from about 1% to about 25% of the crosslinkable component. The polytrimethylene ether polyol is produced via the polymerization of 1,3-propanediol as described herein.

The crosslinking component includes one or more crosslinking agents having at least two isocyanate groups, such as a polyisocyanate crosslinking agent. Any of the conventional aromatic, aliphatic, cycloaliphatic, isocyanates, trifunctional isocyanates and isocyanate functional adducts of a polyol and a diisocyanate can be used. Typically useful diisocyanates are 1,6-hexamethylene diisocyanate, isophorone diisocyanate, 4,4′-biphenylene diisocyanate, toluene diisocyanate, bis cyclohexyl diisocyanate, tetramethylene xylene diisocyanate, ethyl ethylene diisocyanate, 2,3-dimethyl ethylene diisocyanate, 1-methyltrimethylene diisocyanate, 1,3-cyclopentylene diisocyanate, 1,4-cyclohexylene diisocyanate, 1,3-phenylene diisocyanate, 1,5-naphthalene diisocyanate, bis-(4-isocyanatocyclohexyl)-methane and 4,4′-diisocyanatodiphenyl ether.

Typical trifunctional isocyanates include triphenylmethane triisocyanate, 1,3,5-benzene triisocyanate and 2,4,6-toluene triisocyanate. Trimers of diisocyanates also can be used, such as the trimer of hexamethylene diisocyanate.

The relative amount of crosslinking agent used in the coating composition is adjusted to provide a molar equivalent ratio of NCO/(OH+NH) in the range of from about 0.5/1 to about 2/1; in some embodiments, in the range of from about 0.75/1 to about 1.5/1; and in some embodiments, in the range of from about 0.85/1 to about 1.5/1.

In some embodiments, the coating composition includes one or more catalysts to enhance crosslinking of the components during curing. Generally, the coating composition includes in the range of from about 0.005% to about 2%; in some embodiments, in the range of from about 0.01% to about 1%; and in some embodiments, in the range of from about 0.02% to about 0.7% of the catalyst, the percentages being in weight percentages based on the total weight of the crosslinkable and crosslinking component solids. These catalysts can be added to the crosslinkable component. Typical catalysts include, for example: dibutyl tin dilaurate; dibutyl tin Diacetate; dibutyl tin dichloride; dibutyl tin dibromide; triphenyl boron; tetraisopropyl titanate; triethanolamine titanate chelate; dibutyl tin dioxide; dibutyl tin dioctoate; tin octoate; aluminum titanate; aluminum chelates; zirconium chelate; hydrocarbon phosphonium halides such as, ethyl triphenyl phosphonium iodide and other such phosphonium salts; zinc octoate; zinc napthanate; and other catalysts or mixtures thereof known to those skilled in the art.

In some embodiments, the coating composition is a solvent-borne coating composition. Some of the suitable solvents include aromatic hydrocarbons, such as petroleum naphtha or xylenes; esters, such as, butyl acetate, t-butyl acetate, isobutyl acetate or hexyl acetate; and glycol ether esters, such as propylene glycol monomethyl ether acetate. The amount of organic solvent added depends upon the desired solids level as well as the desired amount of VOC of the composition. If desired, the organic solvent may be added to both the crosslinking and crosslinkable components of the coating composition.

The amount of solvent added to the coating composition may be adjusted to provide the composition with a VOC (volatile organic content) in the range of from about 0.12 kg to about 0.78 kg of the solvent per liter of the coating composition.

In some embodiments, the coating composition may also contain conventional additives, such as pigments, stabilizers, rheology control agents, flow agents, and toughening agents. Such additional additives will depend on the intended use of the coating composition. For example, any additives that would adversely affect the clarity of the cured coating will not be included when the composition is used as a clear coating. The foregoing additives may be added to either component or both, depending upon the intended use of the coating composition.

Typical pigments that can be added to the composition include the following: metallic oxides, such as titanium dioxide, zinc oxide, iron oxides of various colors, carbon black; filler pigments, such as talc, china clay, barytes, carbonates, silicates; and a wide variety of organic colored pigments, such as quinacridones, copper phthalocyanines, perylenes, azo pigments, indanthrone blues, carbazoles, such as carbozole violet, isoindolinones, isoindolones, thioindigo reds, benzimidazolinones; metallic flake pigments, such as aluminum flakes, mica flakes, pearlescent flakes, or a combination thereof.

To improve weatherability of the coating, about 0.1 wt % to about 5 wt %; in some embodiments, about 0.5 wt % to about 2.5 wt %; and in some embodiments, about 1wt % to about 2 wt % of ultraviolet light stabilizers screeners, quenchers, and/or antioxidants can be added to the composition, the percentages being based on the total weight of the binder and crosslinking components solids. Typical ultraviolet light screeners and stabilizers include for example, benzophenones, such as hydroxyl dodecycloxy benzophenone, 2,4-dihydroxyl benzophenone, and hydroxyl benzophenones containing sulfonic acid groups; benzoates, such as dibenzoate of diphenylol propane and tertiary butyl benzoate of diphenylol propane; triazines, such as 3,5-dialkyl-4-hydroxyphenyl derivatives of triazine and sulfur containing derivatives of dialkyl-4-hydroxyl phenyl triazine, hydroxyl phenyl-1,3,5-triazine; triazoles, such as 2-phenyl-4-(2,2′-dihydroxyl benzoyl)-triazole and substituted benzotriazoles, such as hydroxy-phenyltriazole; hindered amines, such as bis (1,2,2,6,6-pentamethyl-4-piperidinyl sebacate) and di [4 (2,2,6,6-tetramethyl piperidinyl)]sebacate; and any mixtures of any of the above.

In some embodiments, a coating composition comprising a reactive diluent disclosed herein has a chemical resistance of at least about 20 s, at least about 21 s, at least about 22 s, at least about 23 s, at least about 24 s, at least about 25 s, at least about 26 s, at least about 27 s, at least about 28 s, at least about 29 s, at least about 30 s, at least about 31 s, at least about 32 s, at least about 33 s, at least about 34 s, at least about 35 s, at least about 36 s, at least about 37 s, at least about 38 s, at least about 39 s, at least about 40 s, at least about 41 s, at least about 42 s, at least about 43 s, at least about 44 s, at least about 45 s, at least about 46 s, at least about 47 s, at least about 48 s, at least about 49 s, at least about 50 s, at least about 51 s, at least about 52 s, at least about 53 s, at least about 54 s, at least about 55 s, at least about 56 s, at least about 57 s, at least about 58 s, at least about 59 s, at least about 60 s, at least about 61 s, at least about 62 s, at least about 63 s, at least about 64 s, at least about 65 s, or more as determined by an MEK test.

In some embodiments, the coating composition has a Persoz hardness of at least about 40%, at least about 41%, at least about 42%, at least about 43%, at least about 44%, at least about 45%, at least about 46%, at least about 47%, at least about 48%, at least about 49%, at least about 50%, or more as determined under ISO 1522.

In some embodiments, the coating composition has an impact resistance of at least about 950 mm, at least about 955 mm, at least about 960 mm, at least about 965 mm, at least about 970 mm, at least about 975 mm, at least about 980 mm, at least about 985 mm, at least about 990 mm, at least about 995 mm, at least about 1000 mm, at least about 1005 mm, at least about 1010 mm, at least about 1015 mm, at least about 1020 mm, at least about 1025 mm, or more on a substrate side an object coated with the coating composition as determined by a falling weight test EN ISO 6272-1.

Coating compositions disclosed herein are suitable for providing coatings on a variety of substrates, such as metal, wood, and concrete substrates and resinous surfaces, such as, for example, RIM (reaction injection molded) auto bumpers and dashboards. The present composition is suitable for providing clear or pigmented coatings (i.e., primer compositions or basecoat compositions) in automotive OEM (original equipment manufacturer) applications and refinish applications typically used in making repairs and touch-ups to automotive bodies. The coating compositions can be used in other applications, such as coating truck bodies, boats, airplanes, tractors, cranes, and other metal bodies. The coating compositions are also suitable for use in industrial and maintenance coating applications.

### EXAMPLES

### Example 1

Production of 1,3-propanediol Acrylate (Prophetic)

The mixture is heated to 80° C. and the process is operated at this temperature for approximately 30 minutes with regulation of the pressure, which varies from 2.933×10⁴ Pa (220 mmHg) to 2.266×10⁴ Pa (170 mmHg). Then, at 2.266×10⁴ Pa (170 mmHg), the temperature is allowed to develop until it reaches 100° C. Throughout the reaction the water of reaction is removed by distillation of the hetero-azeotrope. The rate of introduction of the 1,3-propanediol into the decanter is equal to the rate of withdrawal of the water of reaction, the 1,3-propanediol being added over 80 minutes. The total mass of 1,3-propanediol introduced deferredly is introduced when approximately 85% of the expected mass of water of reaction has been drawn off. The reaction is considered over when the acrylic acid content of the reaction medium reaches 0.5%. The crude reaction product is cooled and then neutralized with 8% sodium hydroxide solution in order to remove the catalyst. The organic phase is analysed by gas chromatogarph to determine the 1,3-propanediol acrylate content.

### Example 2

Production of 1,3-propanediol Dimethacrylate (Prophetic)

444 kg of 1,3-propanediol, 3018 kg of methyl methacrylate (MMA), 0.167 kg of N,N′-(diphenyl)-p-phenylenediamine as inhibitor and a mixture of 0.5 kg of lithium amide and 0.25 kg of lithium chloride as catalyst are combined in a stirred tank reactor provided with agitator, steam heater, distillation column and condenser and stirred while passing in air. To stabilize the column, a total of 151 kg of MMA containing 0.24kg of hydroquinone monomethyl ether and 0.016 kg of 4-hydroxy-2,2,6,6-tetramethylpiperidin-1-oxyl in dissolved form are introduced into the column runback. The reactor is heated to a temperature at the bottom of 97° C., with the column initially being operated with total reflux (about 15 minutes). As soon as the temperature at the top of the column drops to below 70° C., the methanol/MMA mixture is taken off at a reflux ratio of 2:1. The MMA stock in the reactor is supplemented by metered addition of equal parts of MMA per part of methanol/MMA mixture taken off. A total of 1320 kg of MMA are thus introduced over a period of 5 hours. After 450 l of methanol/MMA mixture has been taken off, another 0.5 kg of lithium amide is added. Over a period of 8 hours, the reflux ratio is raised to 1.1:1 as a function of the decreasing methanol formation. At a temperature at the bottom of 130° C., the reaction is complete and excess MMA is taken off under reduced pressure, with the pressure gradually being reduced to 100 mbar. When no more MMA distills off, the vacuum is broken. The contents of the tank, comprising the catalyst-containing 1,3-propanediol dimethacrylate, are admixed with 10 kg of aluminum silicate (Perlite) as filter aid and freed of catalyst by washcoat filtration or with the aid of a candle filter.

### Example 3

Production of 1,3-propanediol Diacrylate (Prophetic)

50 parts of 1,3-propanediol and 101 parts of triethylamine are stirred vigorously in a dried ether solvent, and 100 parts of acryloyl chloride is added dropwise gradually at 0° C. to 10° C. while the reaction vessel is cooled from outside with iced water. After dropping, the stirring is continued for five hours at ordinary temperature to confirm the completion of the reaction. Thereafter triethylamine hydrochloride is filtered off from the reaction mixture, and the filtrate is washed with water till it becomes neutral, is dried over anhydrous magnesium sulfate, and is filtered again. Next, the filtrate is distilled under reduced pressure to remove ether, and 102 parts of pale yellow liquid is obtained as residue. The residue in which 5 parts of cuprous chloride is added as a polymerization inhibitor is vacuum distilled to give 90 parts of colorless, transparent liquid (71° C./3 mmHg). The resulting liquid product is identified as 1,3-propanediol diacrylate by means of elementary analysis, infrared absorption spectrum, and nuclear magnetic resonance spectrum.

### Example 4

Production of 1,3-propanediol Diglycidyl Ether (Prophetic)

Step (1), Add 200 g 1,3-propanediol, 1.4 g boron trifluoride-diethyl ether to a 2000 ml four-necked flask, add 472.8 g epichlorohydrin dropwise at 70° C. with stirring, dropwise add 3 hours, keep for 1 hour.

Step (2) add 700 g of toluene, heat up to 40° C. add solid NaOH 204.4 g, take 4 hours, keep for 1 hour, after the reaction is over, transfer to Buchner funnel for suction filtration, add a certain amount of phosphoric acid to the filtrate for neutralization, and then transfer In the separatory funnel for liquid separation, after liquid separation, the toluene solvent is removed to obtain 1,3-propanediol diglycidyl ether.

### Example 5

### Coating Composition

From the broad range of UV curable resins and formulations, Applicant prepared a composition with high concentration of reactive diluent. Coating formulation consists only of acrylic resin, reactive diluent, and photoinitiator. Composition for acrylate and methacrylate diluent differs in initiator concentration a little bit due to expected lower methacrylate reactivity.

TABLE 1
UV coating composition

| | % for acrylate | % for methacrylate |
|---|---|---|
| Acrylate resin | 48.5 | 47.5 |
| Reactive diluent | 48.5 | 47.5 |
| Photoinitiator | 3 | 5 |

### Resin

GENOMER*4215 by RAHN is an aliphatic Polyester Urethane Acrylate. It is a resin for radically curable inks, coatings and adhesives. This product is recommended for use in the following applications: printing inks and varnishes for plastic foils (PVC, PE etc.); PVC flooring; adhesives; and plastic varnishes.

| Tg | −24° | C. |
|---|---|---|
| Viscosity | 20000 | mPas |
| Functionality | 2 | |
| Acid value | 1 | mg KOH/g |

| | |
|---|---|
| Color | 2G |

### Photo-Initiator

GENOCURE*LTM is a non-yellowing photoinitiator blend by Rahn. It shows good balance of surface and thorough cure. Suitable for plastic coating, wood coating, overprint varnishes, flexo-and screen inks.

| | | |
|---|---|---|
| Absorption | 253/368 | nm |
| Viscosity | −20 | cP |
| Viscosity | −20 | mPas |

| | |
|---|---|
| Purity | >97% |

### Reactive Diluents

Acrylates

Four products was chosen for testing:

PDDA 17—esterification route with pTSA

PDDA 13—reesterification route with zinc acetate Biologically produced propane-1,3-diol diacrylate from BCH (Germany)

1,4-BDDA—1,4-butanediol diacrylate from Sigma Aldrich, technical grade, contains ˜75 ppm hydroquinone as inhibitor

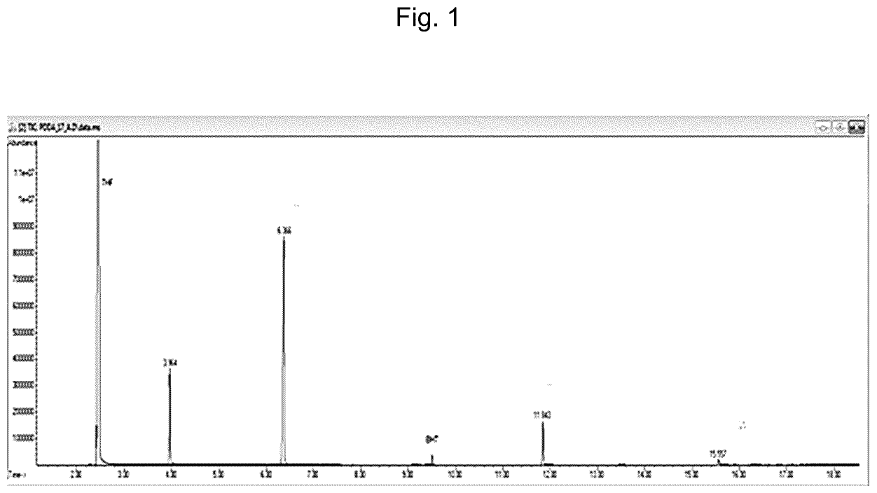

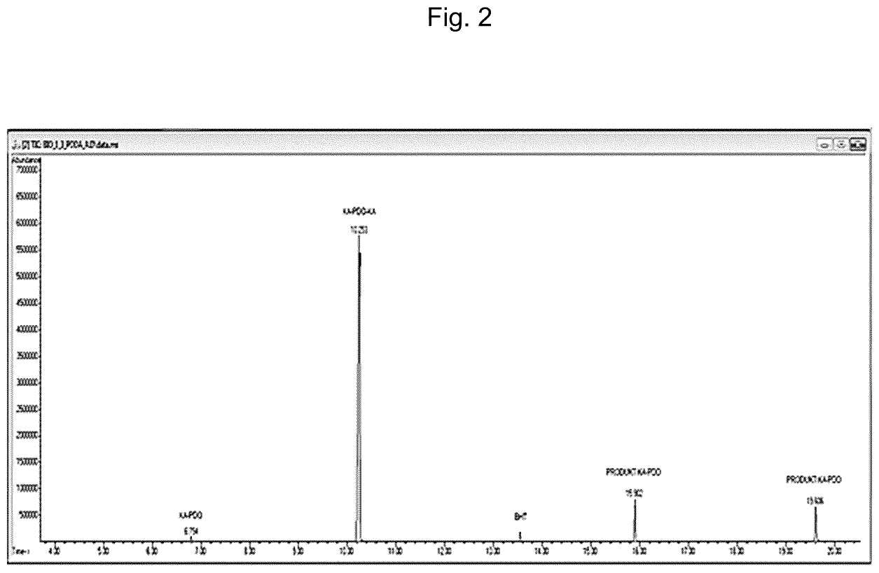

The materials have the characterizations shown in Table 2 and FIG. 1 and FIG. 2

TABLE 2
1,4-butanediol diacrylate from Sigma Aldrich

| Test | Specification | Result |
|---|---|---|
| Appearance (Color) | Colorless | Colorless |
| Appearance (Form) | Liquid | Liquid |
| Infrared Spectrum | Conforms to Structure | Conforms |
| Purity (GG) | ≥87.0% | 89.2% |
| HQ as inhibitor | | 60 ppm |

Specification: Approximately 75 ppm

PDDA 13 from reesterification route with zinc acetate was not tested because it is not stable. Material formed insoluble gel during storage at room temperature.

### Methacrylates

Two products was chosen for testing:

PDDMA 8—vesterification route with pTSA

1,4-BDDMA—1,4-butanediol dimethacrylate from Sigma Aldrich, technical grade, 95%, contains 200-300 ppm MEHQ as inhibitor

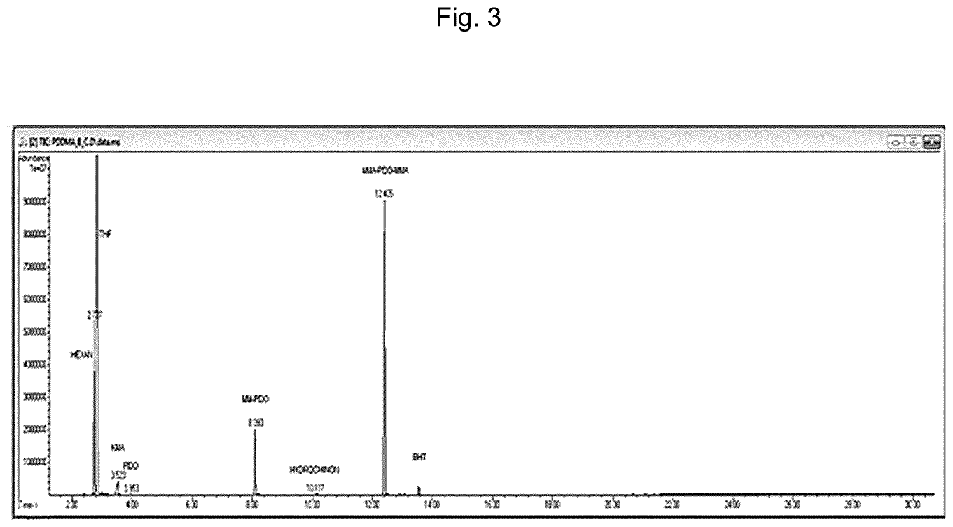

PDDMA has the characterization shown in FIG. 3.

### Coating Preparation

Acrylic resin, reactive diluent and photoinitiator were homogenized using magnetic stirrer. All work was carried out in a darkened room (closed window blinds) to avoid spontaneous polymerization.

Coatings were applied on glass sheets (transparent or black) and on steel panels using frame applicator. Gap size—wet coating thickness—was 50 microns. Transparent glass panels were used for testing of appearance, hardness, microhardness. Black glass was used for gloss measurement and steel panels for mechanical properties determination.

Prepared coatings were cured using compact tabletop UV dryer Miniterm UV 5250f Mf Super by Aerotherm.

TABLE 3

| | |
|---|---|
| Parameters/Type | MINITERM UV 250f Mf Super |

| Belt Width | 250 | mm |
|---|---|---|
| Irradiated Width | 160 | mm |
| Max. Product Height | 100 | mm |
| Conveyor Speed | 2.5-15 | m/min |
| Linear UV Power | switchable 60/120 W/cm | |
| Dryer Dimensions (l × w × h) | 1200 × 450 × 500 | mm |
| Installed Power | 2400 | W |

| | |
|---|---|
| Electric Supply | 1 N PE 230 V |

### Testing Methods

Testing was carried oud in two steps. Preliminary test should verify sufficient conversion after curing-curing conditions. It was evaluated by microhardness measurement. After that, coatings were prepared according optimized curing setup and following parameters were evaluated:

### Appearance

Conversion—FTIR

Persoz hardness

Fisher Hardness—microhardness

Tg—DSC

Adhesion—cross cut test

Impact EN ISO 6272-1

Flexibility

Cupping test Erichsen

Chemical resistance—MEK test

Gloss 20°/60°/80°

UV Energy—radiometer—UV Integrator 150

Measurement of UV irradiance of exposed sample, for a range of wavelengths 250-410 nm with a maximum around 330 nm. The total radiation energy is recorded in mJ/cm².

### Conversion—FTIR

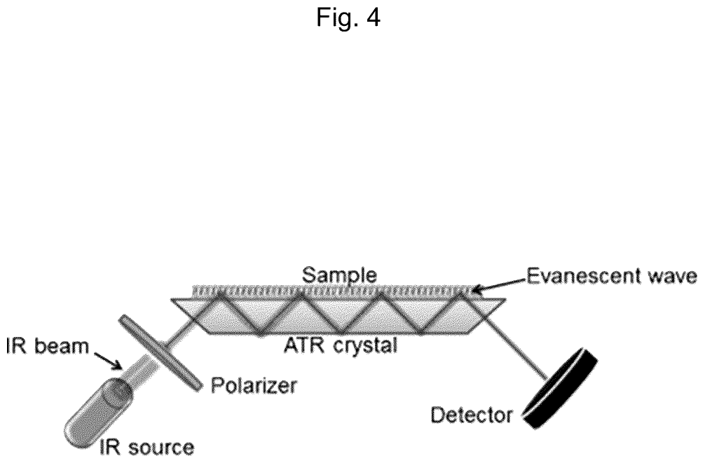

FTIR spectrometer Nicolet (model Impact 400D) in ATR (Attenuated Total Reflectance) mode was used for the analyses. Transmission spectrum of the sample was collected in the middle infrared region (4000-400 cm⁻¹). Decrease of the double bond of acrylates at 810 cm⁻¹ was evaluated. Band of C—H stretching vibrations (2980 cm⁻¹) was used as reference band. See FIG. 4 for an exemplary diagram of a FTIR spectrometer.

### Glass Transition Temperature—Tg—DSC

DSC Q2000 V24.11 Build 124 from TA Instruments from Waters Corporation

Method

Equilibrate at −70° C.

Isothermal for 1.00 min

Ramp 20° C./min to 100.00° C.

Isothermal for 1.00 min

Ramp 20° C./min to −70° C.

Equilibrate at −70° C.

Isothermal for 1.00 min

Ramp 20.00° C./min to 100.00° C.

### Dry Film Thickness

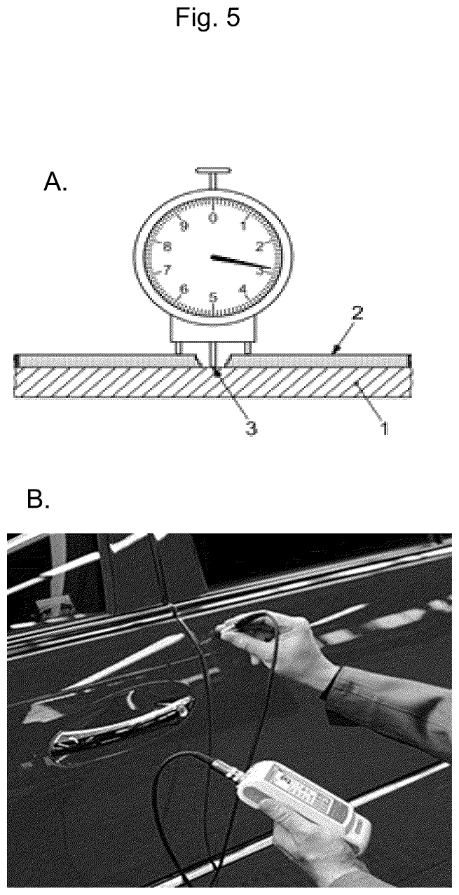

A destructive-dial depth gauge for measurement on glass is shown in FIG. 5A. A non-destructive-magnetic/electromagnetic gauge for measurement on steel is shown in FIG. 5B.

### Persoz Hardness ISO 1522

The Persoz pendulum is rectangular and rests on two tungsten carbide balls, each 8 mm in diameter and 50 mm apart. The total weight of the pendulum is 500 g. The damping period is measured between 12° and 4° deflection. At the 4° position, the count stops. With the Persoz pendulum, there is no counterweight for adjustment, i.e. dimensions, total mass and center of gravity of the pendulum must be very precisely designed to achieve a natural oscillation frequency of 1 sec and a damping period of 430 sec on a flat glass plate. The glass plate is supplied as a reference tile for verification of the measuring system, whereby the mean value of three measurements is to be formed. Table 4 compares Persoz hardness with König hardness.

TABLE 4
Persoz hardness - Pendulum Damping Test, ISO 1522

| | König | | Persoz | |
|---|---|---|---|---|
| Weight | 200 g ± 0.2 | | 500 g ± 0.1 | |
| Diameter | 0.2 in | (5 mm) | 0.3 in | (8 mm) |
| Deflection Start | 6° | | 12° | |
| Deflection End | 3° | | 4° | |
| Period of Oscillation | 1.4 | s | 1 | s |
| Damping Time on Glass | 250 ± 10 | s | 430 ± 10 | s |

### Fisher Hardness—Microhardness

CSN EN ISO 14577-1 Metallic materials-Instrumented indentation test for hardness and materials parameters.

Equipment—FischerScope HM2000S

Conditions—F=2 mN/20 s, C=5 s at 23° C.

### Adhesion-Cross Cut Test-ENISO 2409

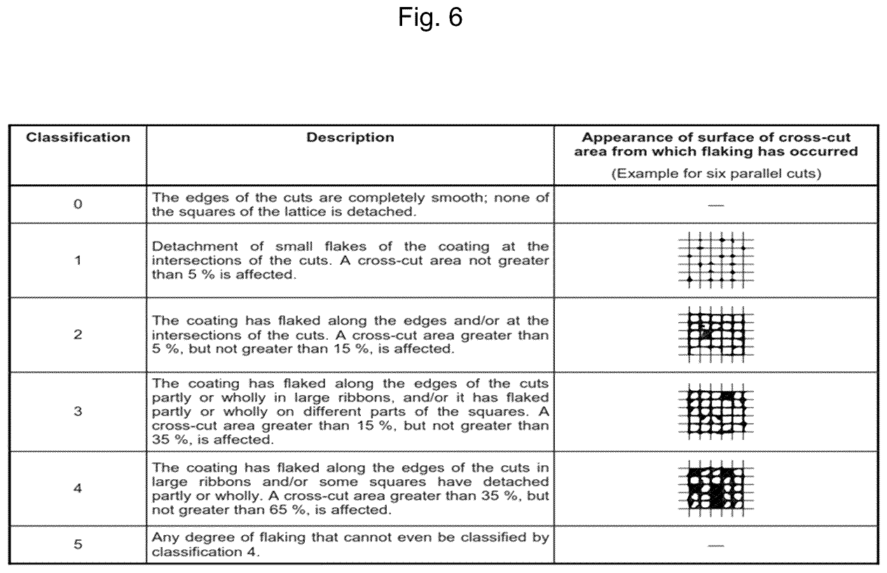

The specifies a test method for assessing a resistance of paint coating to separation from substrates when a right-angle lattice pattern is cut into the coating, penetrating through to the substrate. Description of the ENISO 2409 characterization is shown in FIG. 6.

### Impact Resistance—Falling Weight Test EN ISO 6272-1

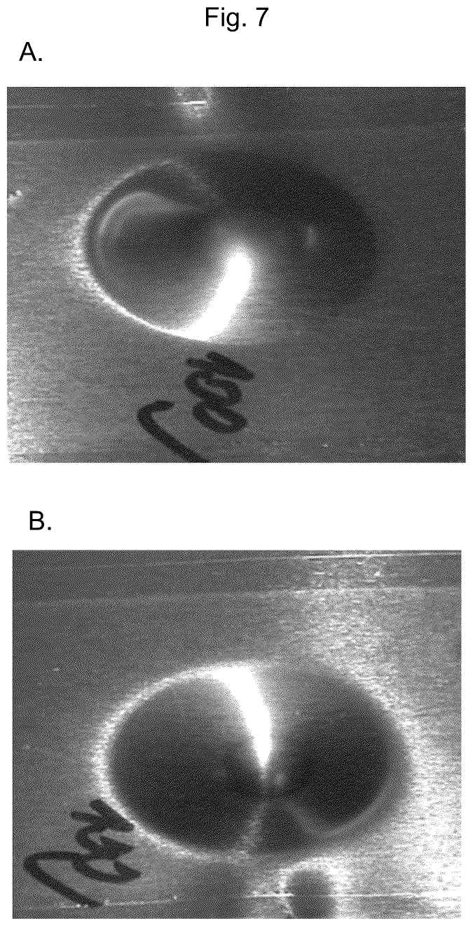

Resistance of a dry film of paint, to cracking or peeling from substrate when it is subjected to a deformation caused by a falling weight with a spherical intender dropped under standard conditions (FIG. 7A shows plates side and FIG. 7B shows coatings side).

### Flexibility—Bend Test—ISO 1519

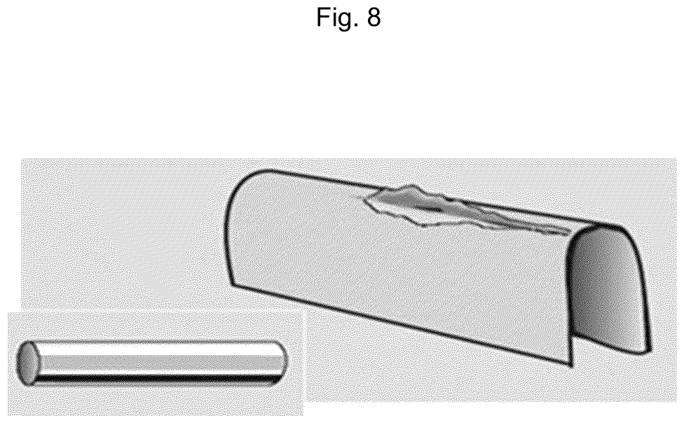

ISO 1519, this test specifies an empirical test procedure for assessing the resistance of a coating of paint, to cracking and/or detachment from a metal substrate when subjected to bending round a cylindrical mandrel under standard condition (exemplary test shown in FIG. 8 with inset).

### Erichsen Cupping Test ISO 1520

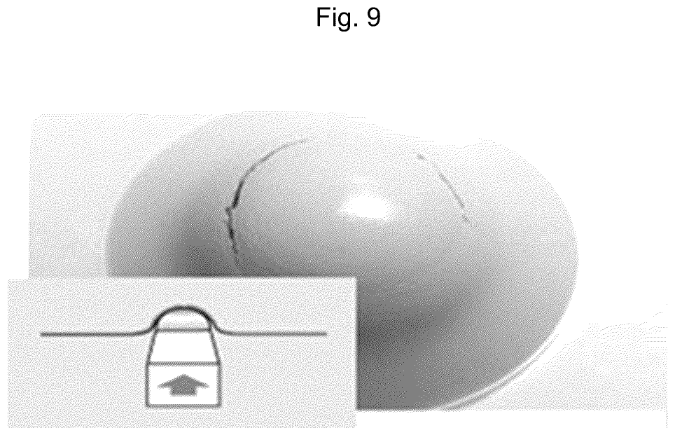

Resistance of a dry film of paint to cracking and/or detachment from a metal substrate when subjected to gradual deformation by indentation under standard conditions (exemplary test shown in FIG. 9 with inset).

### Chemical Resistance—Methyl Ethyl Ketone (MEK) Test

MEK test involves rubbing the surface of a cured film with cloth soaked with MEK until failure or breakthrough of the film occurs. ASTM D4752 involves rubbing the surface of a baked film with cheesecloth soaked with MEK until failure or breakthrough of the film occurs. The type of cheesecloth, the stroke distance, the stroke rate, and approximate applied pressure of the rub are specified. The rubs are counted as a double rub (one rub forward and one rub backward constitutes a double rub).

### Gloss 20°/60°/80°—Micro TRI Gloss (BYK Gardner)—ISO 2813:2014

ISO 2813:2014 specifies a method for determining the gloss of coatings using the three geometries of 20°, 60° or 85°. The method is suitable for the gloss measurement of non-textured coatings on plane, opaque substrates, e.g. black glass panels 100×200×5 mm.

### Results

### Acrylates

The first series of experiment was carried out to assess the reactivity of acrylates that can be different due to various inhibitor levels. Prepared mixtures were coated to glass sheet and cured at various conditions. All systems contained the same initiator content 3% wt and were applied using frame applicator with 50 μm gap.

The aim was to find curing condition leading to maximal conversion. It was evaluated by Persoz hardness and by indentation microhardness.

When the coating were exposed to approx. 3000 mJ/cm² the hardness of composition with biologically-produced propane-1,3-diol diacrylate and 1,4-butanediol diacrylate was about 53% (Persoz) and 55 N/mm² (Indentation) but for PDDA 17 it was only 43% and 41 N/mm² (Table 5). Additional irradiation to 5 000 mJ/cm² increased hardness to 46% and 43 N/mm². Differences between samples can be attributed to higher content of hydroquinone in PDDA 17.

TABLE 5
UV coatings acrylates - preliminary testing

| | Biologically | | | |
|---|---|---|---|---|
| | produced | | | |
| | Propane-1,3-diol | 1,4-butanediol | | |
| Reactive solvent | diacrylate | diacrylate | PDDA 17 | |
| Applicator(um) | 50 | 50 | 50 | 50 |
| Curing regime (time/power) | 3x-12 s/2 | 3x-12 s/2 | 3x-12 s/2 | 5x-12 s/2 |
| UV energy (mJ/cm²) | 3012 | 3028 | 3000 | 4961 |
| Persoz Hardness (%) | 53.4 | 52.5 | 42.9 | 46.1 |
| Microhardness (N/mm²) | 57.4 ± 2.9 | 52.8 ± 2.2 | 41.2 ± 1.7 | 48.3 ± 4.5 |
| Appearance | No defect | No defect | No defect | No defect |

Based on these results, the complete evaluation was carried out in two regimes

3000 mJ/cm² for biologically produced propane-1,3-diol diacrylate and 1,4-butanediol diacrylate

5000 mJ/cm² for PDDA 17

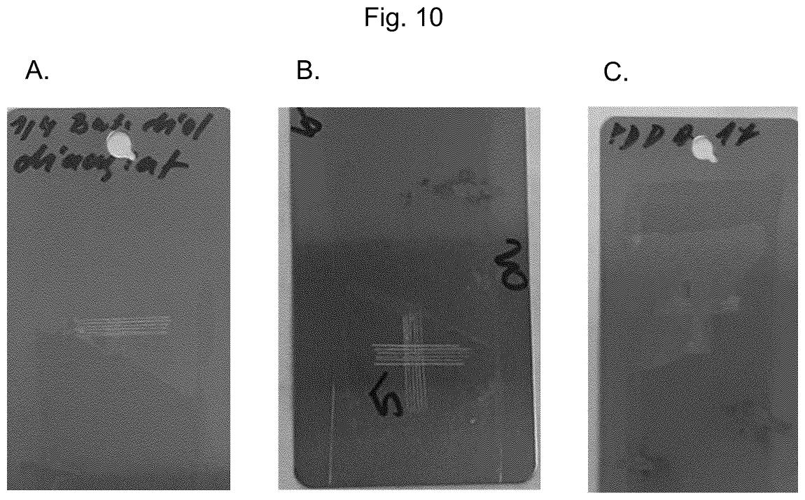

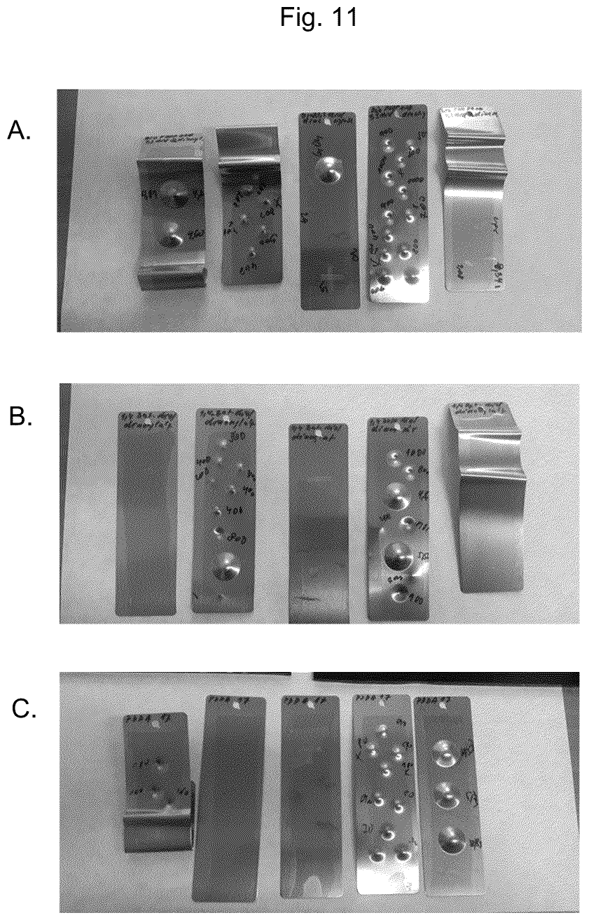

The coatings were applied on glass, black glass and steel panels. Results are summarized in Table 6 and FIGS. 10 and 11.

TABLE 6
UV coatings acrylates - complete testing

| | Biologically | | |
|---|---|---|---|
| | produced- | | |
| | propane- | 1,4- | |
| | 1,3-diol | butanediol | |
| | diacrylate | diacrylate | PDDA 17 |
| Coating preparation | | | |
| Genocure LTM (%) | 3 | 3 | 3 |
| Applicator (μm) | 50 | 50 | 50 |
| Coatings on glass | | | |
| Curing regime (time/power) | 3x-12 s/2 | 3x-12 s/2 | 5x-12 s/2 |
| UV energy (mJ/cm²) | 3337 | 2794 | 5361 |
| Dry film thickness (μm) | 15 | 20 | 18 |
| Appearance | No defect | No defect | No defect |
| Persoz Hardness (%) | 45 | 37 | 42 |
| Microhardness (N/mm²) | 52.9 ± 2.5 | 43.2 ± 0.3 | 43.9 ± 1.0 |
| Gloss 20°/60°/85° | 78.8/86.2/93.1 | 80.1/86.6/93.9 | 69/83/92 |
| Coatings on steel | | | |
| Curing regime (time/power) | 3x-12 s/2 | 3x-12 s/2 | 5x-12 s/2 |
| UV energy (mJ/cm²) | 3318 | 3318 | 5095 |
| Appearance | Clear, few | Clear, few | Clear, few |
| | defects | defects | defects |
| Dry film thickness (μm) | 24.6 | 18.7 | 16.5 |
| Impact resistance (mm) | 1000/300 | 900/300 | >1000/800 |
| substrate side/coating side | | | |
| Flexibility | 5 | 5 | <3 |
| Cupping test Erichsen (mm) | 4.4 | 5.05 | 5.5 |
| Adhesion | 5 | 5 | 5 |
| Chemical resistance (s) | 20-34 | 10-16 | 21-64 |

### Comparison of the Properties Showed Only Small Difference

Comparing to 1,4-BDDA we can state that both 1,3-PDDA are a little bit harder.

PDDA 17 has slightly lower gloss at 20°, higher impact resistance and lower flexibility. It can be associated with lower oligomeric content.

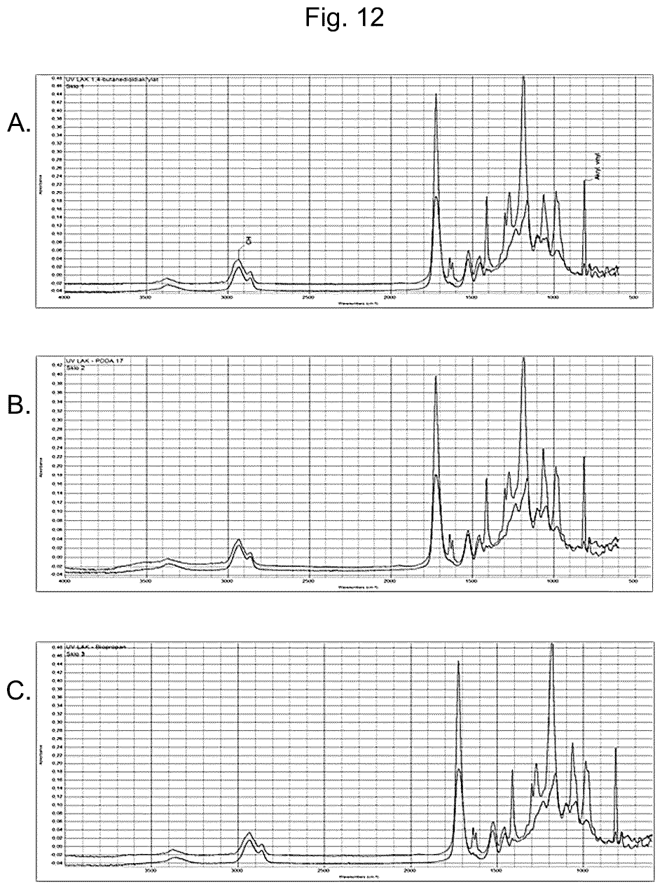

Conversion of the double bonds was measured by infrared spectroscopy and was around 90% for all samples (FIG. 12A—1,4-butandioldiacrylate, FIG. 12B—PDDA 17, FIG. 12C—biologically produced propane-1,3-diol diacrylate).

### Glass Transition Temperature was Measured by DSC

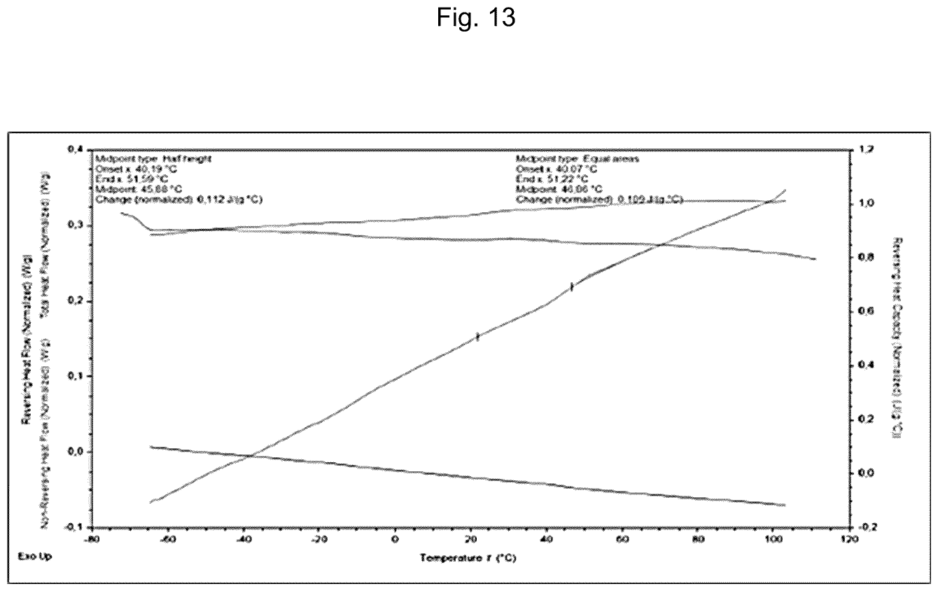

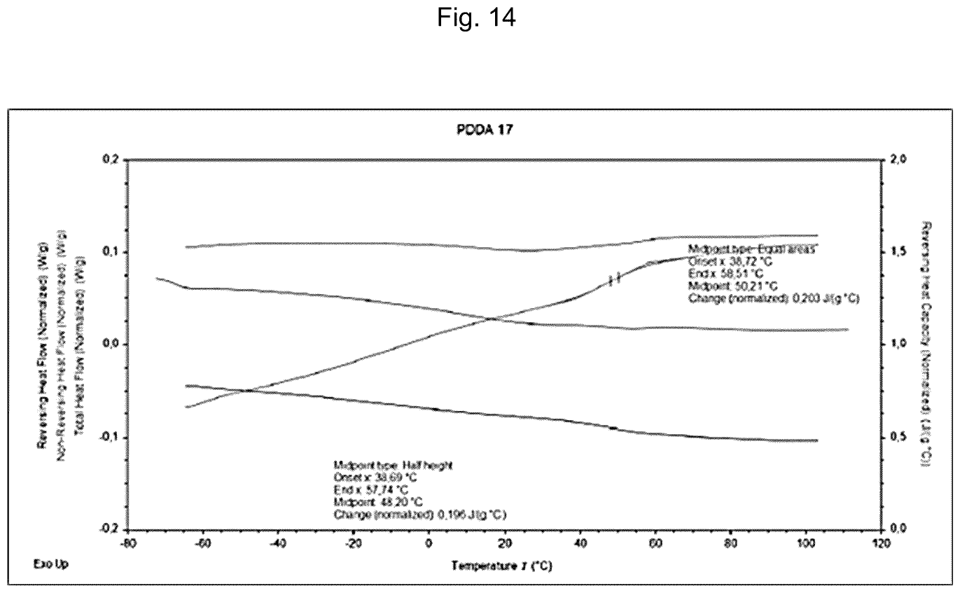

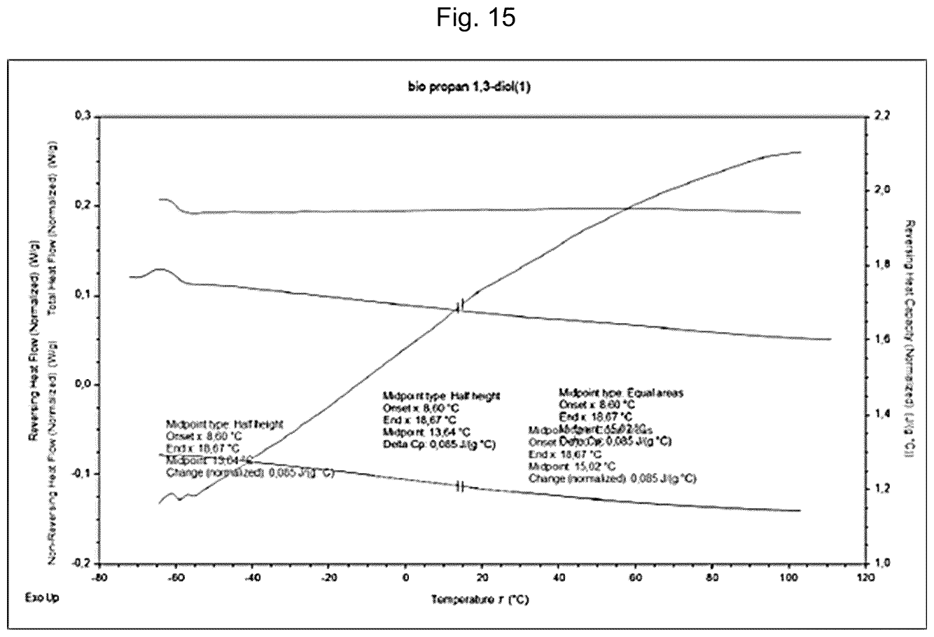

Tg of 1,4-BDDA coating was 51.5° C., PDDA 17 coating 57.7° C. and biologically produced propane-1,3-diol diacrylate only 18.7%. Applicant speculates that it is caused by higher concentration of oligomers or from another reason. Results are shown in Table 7 and FIG. 13 for 1,4-butandioldiacrylate, Table 8 and FIG. 14 for PDDA 17, and Table 9 and FIG. 15 for biologically produced propane-1,3-diol diacrylate.

TABLE 7
1,4-butandioldiacrylate

| | Onset | | End | | | | |
|---|---|---|---|---|---|---|---|
| | temp., | | temp., | | Midpoint, | | Δ cₚ, |
| Tg | ° C. | | ° C. | | ° C. | | JK⁻¹g⁻¹ |
| Half height | −17.59° | C. | −1.99° | C. | −9.77° | C. | 0.135 |
| | | | | | | | J/(g · ° C.) |
| Equal areas | −17.59° | C. | −1.99° | C. | −10.24° | C. | 0.135 |
| | | | | | | | J/(g · ° C.) |
| Half height | 40.19° | C. | 51.59° | C. | 45.88° | C. | 0.112 |
| | | | | | | | J/(g · ° C.) |
| Equal areas | 40.07° | C. | 51.22° | C. | 46.86° | C. | 0.109 |
| | | | | | | | J/(g · ° C.) |

TABLE 8
PDDA 17

| | Onset | | End | | | | |
|---|---|---|---|---|---|---|---|
| | temp., | | temp., | | Midpoint, | | Δ cₚ, |
| Tg | ° C. | | ° C. | | ° C. | | JK⁻¹g⁻¹ |
| Half height | −23.71° | C. | 6.62° | C. | −8.61° | C. | 0.212 |
| | | | | | | | J/(g · ° C.) |
| Equal areas | −23.71° | C. | 6.62° | C. | −8.33° | C. | 0.212 |
| | | | | | | | J/(g · ° C.) |
| Half height | 38.69° | C. | 57.74° | C. | 48.20° | C. | 0.196 |
| | | | | | | | J/(g · ° C.) |
| Equal areas | 38.72° | C. | 58.51° | C. | 50.21° | C. | 0.203 |
| | | | | | | | J/(g · ° C.) |

TABLE 9
Biologically produced propane-1,3-diol diacrylate

| | Onset | End | | |
|---|---|---|---|---|
| | temp., | temp., | Midpoint, | Δ cₚ, |
| Tg | ° C. | ° C. | ° C. | JK⁻¹g⁻¹ |
| Half height | 8.60° C. | 18.67° C. | 13.64° C. | 0.085 |
| | | | | J/(g · ° C.) |
| Equal areas | 8.60° C. | 18.67° C. | 15.02° C. | 0.085 |
| | | | | J/(g · ° C.) |
| Half height | 8.60° C. | 18.67° C. | 13.64° C. | 0.085 |
| | | | | J/(g · ° C.) |
| Equal areas | 8.60° C. | 18.67° C. | 15.02° C. | 0.085 |
| | | | | J/(g · ° C.) |

### Methacrylates

### Two Products Were Chosen for Testing

PDDMA 8—Esterification Route With pTSA

1,4-BDDMA—1,4-butanediol dimethacrylate from Sigma Aldrich, technical grade, 95%, contains 200-300 ppm MEHQ as inhibitor

It is known that methacrylates are less reactive than acrylates, therefore Applicant used higher concentration of initiator Genocure LTM in this testing. Its concentration was 5 wt. %.

Preliminary testing showed similar reactivity of 1,3-propanediol dimethacrylate and 1,4-butanediol dimethacrylate. Final curing was reached after application of UV light energy 5000 mJ/cm² (Table 10). Measurement showed lower hardness of the coating from 1,4-butanediol dimethacrylate.

TABLE 10
UV coatings methacrylates - preliminary testing

| | 1,3-propanediol | 1,4-butanediol |
|---|---|---|
| Reactive solvent | dimethacrylate | dimethacrylate |
| Applicator(μm) | 50 | 50 |
| Curing regime (time/power) | 5x/2 | 5x/2 |
| UV energy (mJ/cm²) | 5168 | 5814 |
| Persoz Hardness (%) | 46.9 | 32.4 |
| Microhardness (N/mm²) | 38.9 ± 1.3 | 18.6 ± 1.3 |
| Appearance | Soft, clear | Soft, clear |

Results of complete testing are in Tabe 11.

TABLE 11
UV coatings methacrylates - complete testing

| | 1,3-propanediol | 1,4-butanediol |
|---|---|---|
| | dimethacrylate | dimethacrylate |
| Coating preparation | | |
| Genocure LTM (%) | 5 | 5 |
| Applicator (μm) | 50 | 50 |
| Coatings on glass | | |
| Curing regime (time/power) | 5x/2 | 4x/2 |
| UV energy (mJ/cm²) | 5295 | 5295 |
| Dry film thickness (μm) | 11 | 20 |
| Appearance | No defect | No defect |
| Persoz Hardness (%) | 41.8 ± 2.5 | 35.6 ± 0.9 |
| Microhardness (N/mm²) | 34.8 ± 1.2 | 25.5 ± 0.6 |
| Gloss 20°/60°/85° | 51.9/ | 77.1/ |
| | 78.5/95 | 87.1/95.1 |
| Coatings on steel | | |
| Curing regime (time/power) | 5x/2 | 4x/2 |
| UV energy (mJ/cm²) | 5295 | 5295 |
| Dry film thickness (μm) | 18.6 | 14.2 |
| Impact resistance (mm) | 800/<100 | 400/<100 |
| substrate side/coating side | | |
| Flexibility | 5 | 10 |
| Cupping test Erichsen (mm) | 4.3 | 2.7 |
| Adhesion | 5 | 5 |

| | |
|---|---|
| Chemical resistance | Scattered results due to poor adhesion |

Coatings containing 1,3-propanediol dimethacrylate exhibit higher hardness and impact resistance and lower flexibility. Adhesion to glass and steel is poor. It affects determination of chemical adhesion, because small swelling of the coating cause loss of the adhesion prior the damage of the surface by solvent.

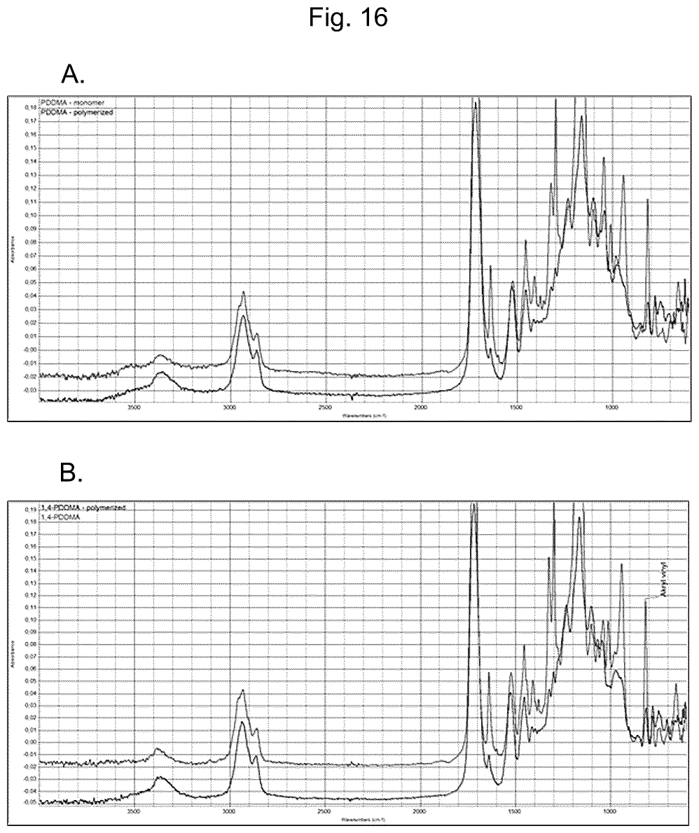

Conversion of the double bonds was similar for both types of binders (see FIG. 16A for 1,3-propanedioldimethacrylate and FIG. 16B for 1,4-butanediol dimethacrylate).

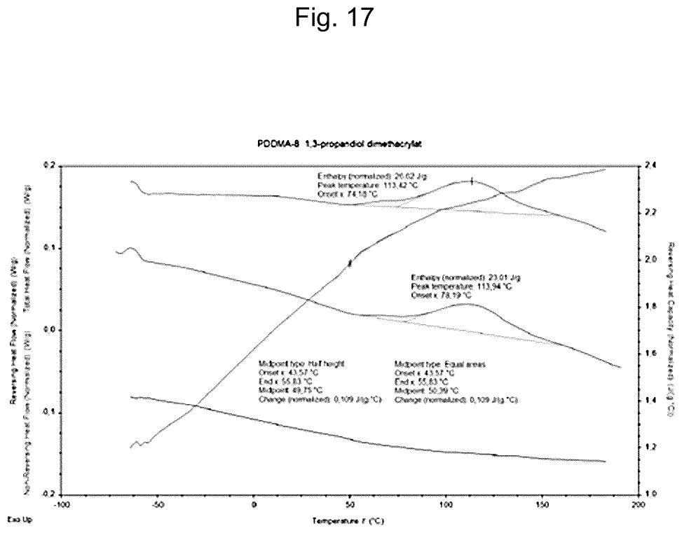

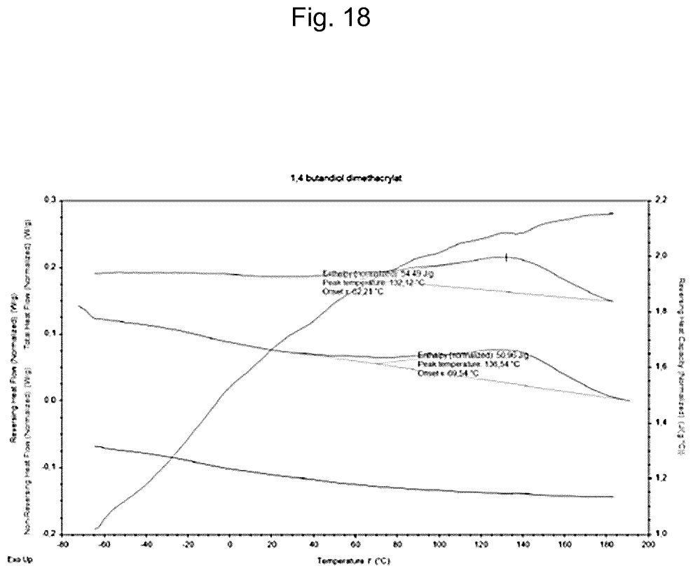

Tg was measured by DSC. It is 50° C. for 1,3-propanedioldimethacrylate, but Applicant was not able to determine it for 1,4-butanediol dimethacrylate products. Results are shown in Table 12 and FIG. 17 for 1,3-propanedioldimethacrylate and Table 13 and FIG. 18 for 1,4-butanediol dimethacrylate.

TABLE 12

| | Onset | End | | |
|---|---|---|---|---|
| | temp., | temp., | Midpoint, | Δ cₚ, |
| Tg | ° C. | ° C. | ° C. | JK⁻¹g⁻¹ |
| Half Height | 43.57° C. | 55.83° C. | 49.75° C. | 0.109 J/(g · ° C.) |
| Equal areas | 43.57° C. | 55.83° C. | 50.39° C. | 0.109 J/(g · ° C.) |

| | Onset | Peak | |
|---|---|---|---|
| | temp., | temp., | |
| ΔH | ° C. | ° C. | Enthalpy |
| Non- | 74.18° C. | 113.42° C. | −26.62 J/g |
| reversing | | | |
| heat flow | | | |
| Reversing | 78.19° C. | 113.94° C. | −23.01 J/g |
| heat flow | | | |

TABLE 13

| | Onset | End | | |
|---|---|---|---|---|
| | temp., | temp., | Midpoint, | Δ cₚ, |
| Tg | ° C. | ° C. | ° C. | JK⁻¹g⁻¹ |
| Half Height | ? | ? | ? | ? |
| Equal areas | ? | ? | ? | ? |

| | Onset | Peak | |
|---|---|---|---|
| | temp., | temp., | |
| ΔH | ° C. | ° C. | Enthalpy |
| Non- | 62.21° C. | 132.12° C. | −54.49 J/g |
| reversing | | | |
| heat flow | | | |
| Reversing | 69.54° C. | 136.54° C. | −50.96 J/g |
| heat flow | | | |

### Coatings on Wood

Because poor adhesion to steel and glass influences determination of the chemical resistance we decided to prepare coatings on wood. This substrate is better because Genomer 4215 is good for wood coatings (See Rahn Group website).

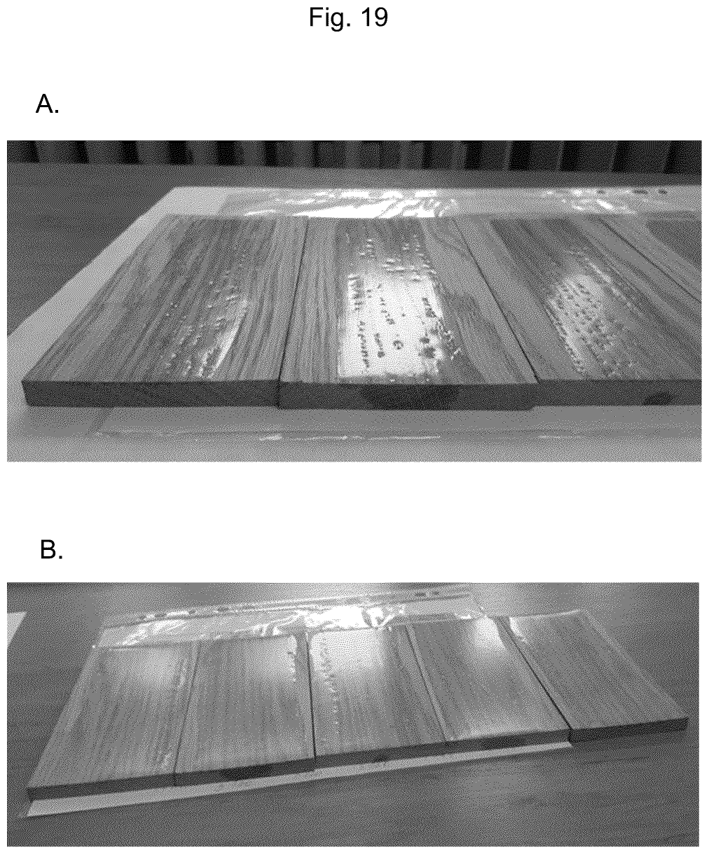

The coatings were applied by gap applicator with gap size 50 μm. Beech wood was used as a substrate. This thickness was not sufficient because in the darker parts of wood it penetrated inside and surface was not uniform (see FIG. 19A).

After the application of the second layer, the surface was nearly smooth for all tested products (see FIG. 19B).

We measured gloss, adhesion and chemical resistance of these samples (Table 14).

TABLE 14
UV coatings on the wood substrate

| | Biologically | | | | |
|---|---|---|---|---|---|
| | produced | | | | |
| Reactive | propane-1,3-diol | 1,4-butanediol | | 1,3-propanediol | 1,4-butanediol |
| solvent | diacrylate | diacrylate | PDDA 17 | dimethacrylate | dimethacrylate |
| Genocure | 3 | 3 | 3 | 5 | 5 |
| LTM (%) | | | | | |
| 1^{st} layer - | 50 | 50 | 50 | 50 | 50 |
| applicator | | | | | |
| (μm) | | | | | |
| Curing regime | 3x/2 | 3x/2 | 5x/2 | 5x/2 | 5x/2 |
| UV energy | 3006 | 3006 | 5140 | 5140 | 5140 |
| (mJ/cm²) | | | | | |
| 2^{nd} layer - | 100 | 100 | 100 | 100 | 100 |
| applicator | | | | | |
| (μm) | | | | | |
| Gloss | 27/68.7/78.7 | 28.2/69.6/77.2 | 26.1/61.4/73.4 | 32.8/72.5/81.8 | 55.1/84.4/87.9 |
| 20°/60°/85° | | | | | |
| Adhesion | 1 | 1 | 1 | 2 | 2 |
| Chemical | >600/495/375/342 | 420/150/175/134 | 139/155/85/133 | 147/94/92/126 | 103/94/95/140 |
| resistance(s) | | | | | |

### Gloss

Coatings have lower gloss than on the glass. While difference between acrylates is negligible, 1,3-propanediol dimethacrylate is less glossy than 1,4-butanediol dimethacrylate.

### Adhesion

Adhesion to wood is much better than on the glass and steel. Acrylates have better adhesion than methacrylates.

### Chemical Resistance

Although the results are still quite scattered, we can say that 1,3-propanediol diacrylate provides better chemical resistance than 1,4-butanediol diacrylate. Difference between methacrylates is low.

### Conclusions

Applicant concludes that prepared 1,3-propanediol diacrylate and 1,3-propanediol dimethacrylate can be used in UV coatings as reactive diluent. Diacrylates prepared by esterification route are stable during storage contrary to product from reesterification process and thus better for UV coatings.

Coatings containing reactive diluent from 1,3-propanediol have slightly higher hardness, impact resistance, and chemical resistance than coatings containing reactive diluent from 1,4-butanediol.

### Example 6

Propanediol diglycidyl ether (PDODGE) was produced by reacting epichlorohydrin and biologically-produced 1,3-propanediol in a reactor in the presence of boron trifluoride diethyl etherate, demineralized water, monosodium phosphate, sodium hydroxide, and toluene. Neat resin (standard with butanediol diglycidyl ether (BDDGE) as the reactive diluent (20%) and test samples with PDODGE as the reactive diluent (20%)) were produced by mixing CHS Epoxy 520 (epoxy component) and Telait 542 (amine component) in a 100:32 and adding reactive diluent. The mixture was cured for 24 hours at 23° C. followed by 2 hours at 120° C. Composites were formed from the resins at a resin density of 1.162 by curing the resins in a 500 g mold. Composite fabric was produced as plain weave in a 50/50 ratio with E-glass at a fiber density of 2.54.

Physical properties of BDDGE and PDODGE composites were tested for Interlaminar shear strength (ILSS), flexural strength, flexural modulus, tensile strength, and tensile modulus.

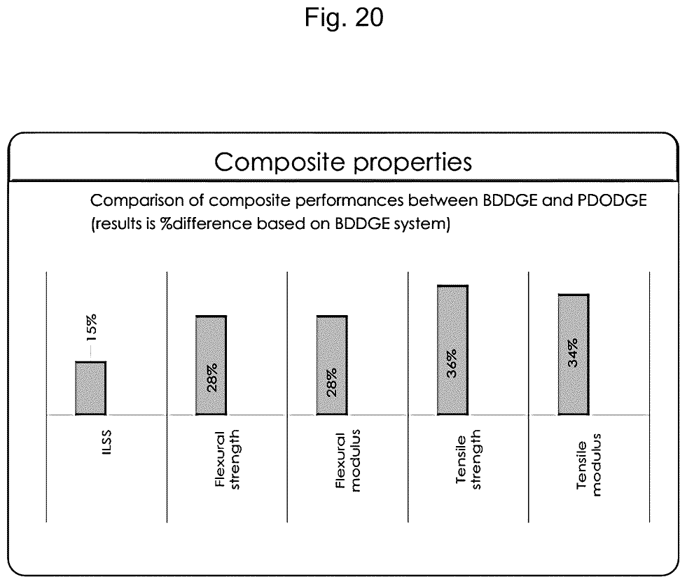

FIG. 20 shows the comparison between baseline composite performance of BDDGE versus biologically-produced PDODGE of the present disclosure. Biologically-produced PDODGE was superior to BDDGE in all physical characteristics tested.

What is claimed is:

1. A reactive diluent comprising a 1,3-propanediol diacrylate component, 1,3-propanediol monoacrylate component, and/or a 1,3-propanediol di-glycidylether component.

2. A coating composition comprising the reactive diluent of claim 1, a resin, and a reaction initiator.

3. The coating composition of claim 2, wherein the resin is an acrylic resin.

4. The coating composition of claim 3, wherein the acrylic resin is a urethane acrylate.

5. The coating composition of claim 2, wherein the reaction initiator is a photoinitiator.

6. A coated article comprising the coating composition of claim 2 on at least of portion of one surface of the article.

7. The coated article of claim 6, wherein the coated article is a metal coated article or a wood coated article.

8. The coating composition of claim 2, wherein the coating composition has a chemical resistance of at least about 20 s.

9. The coating composition of claim 2, wherein the coating composition has a Persoz hardness of at least about 40%.

10. The coating composition of claim 2, wherein the coating composition has an impact resistance of at least about 950 mm on a substrate side an object coated with the coating composition.

11. A process for preparing a coating composition comprising:
(a) mixing a resin with a reactive diluent comprising a 1,3-propanediol diacrylate component, 1,3-propanediol monoacrylate component, and/or a 1,3-propanediol di-glycidylether component; and
(b) reacting the resin with the reactive diluent in the presence of a reaction initiator.

12. The process of claim 11, wherein the resin is an acrylic resin.

13. The process of claim 12, wherein the acrylic resin is a urethane acrylate.

14. The process of claim 11, wherein the reaction initiator is a photoinitiator.

15. A process for coating an article comprising:
(a) mixing a resin, a reaction initiator, and a reactive diluent comprising a 1,3-propanediol diacrylate component, 1,3-propanediol monoacrylate component, and/or a 1,3-propanediol di-glycidylether component to produce a coating mixture;
(b) applying the coating mixture to at least a portion of one surface of an article; and
(c) curing the coating mixture to form a cured coating layer on the article.

16. The process of claim 15, wherein the resin is an acrylic resin.

17. The process of claim 16, wherein the acrylic resin is a urethane acrylate.

18. The process of claim 15, wherein the reaction initiator is a photoinitiator.

19. A process for preparing 1,3-propanediol acrylate comprising:
(a) reacting acrylic acid and 1,3-propanediol in a reactor in the presence of an esterification catalyst, with water formed by the reaction being entrained by distillation in a column in the form of a heteroazeotropic mixture with 1,3-propanediol;
(b) condensing the heteroazeotropic mixture of step (a);
(c) separating the condensed heteroazeotropic mixture of step (b) in a decanter thereby yielding an upper organic phase and a lower aqueous phase;
(d) optionally recycling the upper organic phase to the top of the distillation column; and
(e) drawing off the lower aqueous phase.

20. A process for preparing 1,3-propanediol dimethacrylate comprising transesterifying 1,3-propanediol with an ester of methacrylic acid in the presence of at least one catalyst comprising LiNH₂ and LiCl.

21. A process for preparing 1,3-propanediol diacrylate comprising:
(a) mixing 1,3-propanediol with triethylamine;
(b) adding acryloyl chloride to the mixture of step (a) to produce triethylamine hydrochloride;
(c) filtering the triethylamine hydrochloride from the mixture of step (b) to produce a filtrate without triethylamine hydrochloride;
(d) drying the filtrate of step (c) over anhydrous magnesium sulfate;
(e) distilling the filtrate of step (d) under reduced pressure thereby removing ether and producing a distilled filtrate; and
(f) vacuum distilling the distilled filtrate in the presence of cuprous chloride to produce 1,3-propanediol diacrylate.

22. A process for preparing 1,3-propanediol diglycidyl ether comprising:
(a) reacting epichlorohydrin and 1,3-propanediol in a reactor in the presence of a catalyst and toluene;
(b) dehydrochlorinating the product of step (a);
(c) filtering the dehydrochlorinated product of step (b);
(d) neutralizing the filtered product of step (c);
(e) distilling the neutralized product of step (d); and
(f) filtering the distilled product of step (e).

23. A composite comprising a 1,3-propanediol di-glycidylether component.
